SEQUENCE LISTING (36 sequences)

SEQ ID NO: 1 - DNA, 28 bp, synthetic construct
  misc_feature 1..28: note = Description of Artificial Sequence: Synthetic oligonucleotide
  source 1..28: mol_type = other DNA
taggggccggggccggggccggggcgtg

SEQ ID NO: 2 - DNA, 28 bp, synthetic construct
  misc_feature 1..28: note = Description of Artificial Sequence: Synthetic oligonucleotide
  source 1..28: mol_type = other DNA
cacgccccggccccggccccggccccta

SEQ ID NO: 3 - protein, 7 aa, synthetic construct
  REGION 1..7: note = Description of Artificial Sequence: Synthetic peptide
DRSDLSR

SEQ ID NO: 4 - protein, 7 aa, synthetic construct
  REGION 1..7: note = Description of Artificial Sequence: Synthetic peptide
RSTHLVR

SEQ ID NO: 5 - protein, 7 aa, synthetic construct
  REGION 1..7: note = Description of Artificial Sequence: Synthetic peptide
RSAHLSR

SEQ ID NO: 6 - protein, 7 aa, synthetic construct
  REGION 1..7: note = Description of Artificial Sequence: Synthetic peptide
ERGDLKR

SEQ ID NO: 7 - protein, 7 aa, synthetic construct
  REGION 1..7: note = Description of Artificial Sequence: Synthetic peptide
ERGTLAR

SEQ ID NO: 8 - protein, 7 aa, synthetic construct
  REGION 1..7: note = Description of Artificial Sequence: Synthetic peptide
RSADLSE

SEQ ID NO: 9 - protein, 7 aa, synthetic construct
  REGION 1..7: note = Description of Artificial Sequence: Synthetic peptide
RSDHLSE

SEQ ID NO: 10 - protein, 7 aa, synthetic construct
  REGION 1..7: note = Description of Artificial Sequence: Synthetic peptide
DRSHLAR

SEQ ID NO: 11 - protein, 7 aa, synthetic construct
  REGION 1..7: note = Description of Artificial Sequence: Synthetic peptide
RSDHLSQ

SEQ ID NO: 12 - protein, 7 aa, synthetic construct
  REGION 1..7: note = Description of Artificial Sequence: Synthetic peptide
DNSHRTR

SEQ ID NO: 13 - protein, 7 aa, synthetic construct
  REGION 1..7: note = Description of Artificial Sequence: Synthetic peptide
RNGHLLD

SEQ ID NO: 14 - protein, 7 aa, synthetic construct
  REGION 1..7: note = Description of Artificial Sequence: Synthetic peptide
RSAHLSE

SEQ ID NO: 15 - protein, 7 aa, synthetic construct
  REGION 1..7: note = Description of Artificial Sequence: Synthetic peptide
RSDHLSR

SEQ ID NO: 16 - protein, 7 aa, synthetic construct
  REGION 1..7: note = Description of Artificial Sequence: Synthetic peptide
DWTTRRR

SEQ ID NO: 17 - protein, 7 aa, synthetic construct
  REGION 1..7: note = Description of Artificial Sequence: Synthetic peptide
HRKSLSR

SEQ ID NO: 18 - protein, 7 aa, synthetic construct
  REGION 1..7: note = Description of Artificial Sequence: Synthetic peptide
DSSDRKK

SEQ ID NO: 19 - protein, 7 aa, synthetic construct
  REGION 1..7: note = Description of Artificial Sequence: Synthetic peptide
DSSTRRR

SEQ ID NO: 20 - protein, 7 aa, synthetic construct
  REGION 1..7: note = Description of Artificial Sequence: Synthetic peptide
RSDDRKT

SEQ ID NO: 21 - protein, 7 aa, synthetic construct
  REGION 1..7: note = Description of Artificial Sequence: Synthetic peptide
RSADRKT

SEQ ID NO: 22 - protein, 7 aa, synthetic construct
  REGION 1..7: note = Description of Artificial Sequence: Synthetic peptide
RNADRIT

SEQ ID NO: 23 - protein, 7 aa, synthetic construct
  REGION 1..7: note = Description of Artificial Sequence: Synthetic peptide
RRATLLD

SEQ ID NO: 24 - protein, 7 aa, synthetic construct
  REGION 1..7: note = Description of Artificial Sequence: Synthetic peptide
RSDTLSV

SEQ ID NO: 25 - protein, 7 aa, synthetic construct
  REGION 1..7: note = Description of Artificial Sequence: Synthetic peptide
DTSTRTK

SEQ ID NO: 26 - protein, 7 aa, synthetic construct
  REGION 1..7: note = Description of Artificial Sequence: Synthetic peptide
RSATLSE

SEQ ID NO: 27 - protein, 7 aa, synthetic construct
  REGION 1..7: note = Description of Artificial Sequence: Synthetic peptide
HHRSLHR

SEQ ID NO: 28 - protein, 7 aa, synthetic construct
  REGION 1..7: note = Description of Artificial Sequence: Synthetic peptide
TSSDRTK

SEQ ID NO: 29 - protein, 7 aa, synthetic construct
  REGION 1..7: note = Description of Artificial Sequence: Synthetic peptide
DRSHLTR

SEQ ID NO: 30 - protein, 7 aa, synthetic construct
  REGION 1..7: note = Description of Artificial Sequence: Synthetic peptide
DSSTRKT

SEQ ID NO: 31 - protein, 7 aa, synthetic construct
  REGION 1..7: note = Description of Artificial Sequence: Synthetic peptide
DKRDLAR

SEQ ID NO: 32 - protein, 7 aa, synthetic construct
  REGION 1..7: note = Description of Artificial Sequence: Synthetic peptide
ERRDLRR

SEQ ID NO: 33 - protein, 10 aa, synthetic construct
  REGION 1..10: note = Description of Artificial Sequence: Synthetic peptide
LRQKDAARGS

SEQ ID NO: 34 - protein, 9 aa, unidentified
  REGION 1..9: note = Description of Unknown: "LAGLIDADG" family peptide motif sequence
LAGLIDADG

SEQ ID NO: 35 - DNA, 30 bp, unidentified
  misc_feature 1..30: note = Description of Unknown: G4C2 repeat sequence
  source 1..30: mol_type = other DNA
ggggccggggccggggccggggccggggcc

SEQ ID NO: 36 - DNA, 12 bp, Homo sapiens
  source 1..12: mol_type = other DNA
ggggccggggcc

(19) United States
(12) Patent Application Publication
(10) Pub. No.: US 2025/0099624 A1
(43) Pub. Date: Mar. 27, 2025

(54) METHODS AND COMPOSITIONS FOR THE TREATMENT OF RARE DISEASES

(71) Applicant: Sangamo Therapeutics, Inc., Richmond, CA (US)

(72) Inventors: Michael C. HOLMES, Richmond, CA (US); Brigit E. RILEY, Richmond, CA (US); Thomas WECHSLER, Richmond, CA (US); Bryan ZEITLER, Richmond, CA (US); Lei ZHANG, Richmond, CA (US)

(73) Assignee: Sangamo Therapeutics, Inc., Richmond, CA (US)

(21) Appl. No.: 18/609,899

(22) Filed: Mar. 19, 2024

Related U.S. Application Data
(63) Continuation of application No. 16/169,420, filed on Oct. 24, 2018, now abandoned.
(60) Provisional application No. 62/576,584, filed on Oct. 24, 2017.

(51) Int. Cl. A61K 48/00; A61K 9/00; A61K 31/00; A61K 38/00; A61P 21/00; A61P 25/28; C07K 14/47; C12N 9/22

(52) U.S. Cl. CPC A61K 48/0066; A61K 9/0019; A61K 9/0043; A61K 9/0085; A61K 31/00; A61K 48/0008; A61K 48/005; A61K 48/0058; A61K 48/0075; A61K 48/0091; A61P 21/00; A61P 25/28; C07K 14/4702; C12N 9/22; A61K 38/00; C07K 2319/00; C07K 2319/50; C07K 2319/71; C07K 2319/81; C12N 2750/14143

(57) ABSTRACT

The present disclosure is in the field of modulation of genes involved in rare diseases including for diagnostics and therapeutics for rare diseases such as Angelman's Syndrome, Facioscapulohumeral Muscular Dystrophy (FHMD), Amyotrophic Lateral Sclerosis (ALS), Frontotemporal dementia (FTD) and Spinal Muscular Atrophy (SMA).

### CROSS-REFERENCE TO RELATED APPLICATIONS

The present application is a continuation of U.S. application Ser. No. 16/169,420 filed Oct. 24, 2018, which claims the benefit of U.S. Provisional Application No. 62/576,584, filed Oct. 24, 2017, the disclosures of each of which are hereby incorporated by reference in their entireties.

### SEQUENCE LISTING

The instant application contains a sequence listing which has been submitted electronically in XML format and is hereby incorporated by reference in its entirety. Said XML file, created on Mar. 15, 2024, is named PC202063.XML and is 45400 bytes in size.

### TECHNICAL FIELD

The present disclosure is in the field of diagnostics and therapeutics for rare diseases.

### BACKGROUND

Many, perhaps most physiological and pathophysiological processes can be associated by the aberrant up or down regulation of gene expression. Examples include the inappropriate expression of proinflamatory cytokines in rheumatoid arthritis, under expression of the hepatic LDL receptor in hypercholesteremia, over expression of proangiogenic factors and under expression of antiangiogenic factors in solid tumor growth, to name just a few. In addition, pathogenic organisms such as viruses, bacteria, fungi, and protozoa could be controlled by altering gene expression.

Promoter regions of genes typically comprise proximal, core and downstream elements, and transcription can be regulated by multiple enhancers. These sequences contain multiple binding sites for a variety of transcription factors and can activate transcription independent of location, distance or orientation with respect to the promoter sequence. In order to achieve gene expression regulation, enhancer-bound transcription factors loop out the intervening sequences and contact the promoter region. In addition, activation of eukaryotic genes can require de-compaction of the chromatin structure, which can be carried out by recruitment of histone modifying enzymes or ATP-dependent chromatin remodeling complexes such that chromatin structure is altered and the accessibility of the DNA to other proteins involved in gene expression is increased (Ong and Corces (2011) Nat Rev Genetics 12:283). DNA methylation can also be a factor in the regulation of gene expression. For example, cytosines in the DNA strand can become methylated to become 5-methyl cytosine, and this can occur at a high frequency when cytosines are present in next to a guanine (also known as a “CpG” configuration). In fact, high concentrations of CpGs in promoter regions, so-called CpG islands, are often methylated or demethylated to regulate promoter function (see Lister et al (2009) Nature 462(7271):315-22).

Perturbation of chromatin structure can occur by several mechanisms-some which are localized for a specific gene, and others that are genome wide and occur during cellular processes such as mitosis where condensation of the chromatin is required. Lysine residues on histones may become acetylated, effectively neutralizing the charge interaction between the histone proteins and the chromosomal DNA. This has been observed at the hyperacetylated and highly transcribed β-globin locus which has also been shown to be DNAse sensitive, a hallmark of general accessibility. Other types of histone modifications that have been observed include methylation, phosphorylation, deamination, ADP ribosylation, addition of β-N-acetlyglucosamine sugars, ubiquitylation and sumoylation (see Bannister and Kouzarides (2011) Cell Res 21:381). It also appears that DNA methylation can also impact histone modification. In some instances, methylated DNA is associated with increased histone modification, leading to a more condensed form of chromatin (Cedar and Bergman (2009) Nature Rev Gene 10:295-304).

Repression or activation of disease associate genes has been accomplished through the use of engineered transcription factors. Methods of designing and using engineered zinc finger transcription factors (ZFP-TF) are well documented (see for example U.S. Pat. No. 6,534,261), and more recently both transcription activator like effector transcription factors (TALE-TF) and clustered regularly interspaced short palindromic repeat Cas based transcription factors (CRISPR-Cas-TF) have also been described (see review Kabadi and Gersbach (2014) Methods 69(2): 188-197). Non-limiting examples of targeted genes include phospholamban (Zhang et al (2012) Mol Ther 20(8): 1508-1515), GDNF (Langaniere et al (2010) J. Neurosci 39(49): 16469) and VEGF (Liu et al (2001) J Biol Chem 276:11323-11334). In addition, activation of genes has been achieved by use of a CRIPSR/Cas-acetyltransferase fusion (Hilton et al (2015) Nat Biotechnol 33(5):510-517). Engineered TFs that repress gene expression (repressors) have also been shown to be effective in modulating genes involved in trinucleotide disorders such as Huntingtin's Disease (HD) and in tauopathies. See, e.g., U.S. Pat. Nos. 9,234,016; 8,841,260; and 8,956,8282 and U.S. Patent Publication Nos. 20180153921 and 20150335708. In addition, gene expression may be regulated by engineered nucleases (e.g. zinc finger nucleases, TALE nucleases, CRISPR/Cas systems and the like), where the gene is specifically cleaved by the engineered nuclease. Error-prone repair of the cleavage site often results in insertions and deletions of nucleotides (“indels”), which will cause a knock-out of gene expression.

Rare diseases can often be devastating for patients and their families. For example, Angelman's Syndrome, Facioscapulohumeral Muscular Dystrophy (FHMD), Spinal Muscular Atrophy (SMA), and the c9Orf72 implications in Amyotrophic Lateral Sclerosis (ALS) and familial frontotemporal dementia (FTD) are all diseases that can have lifelong effects, such as mental retardation (Angelman's Syndrome), cognitive deficits (e.g., FTD) and/or muscle debilitation (FHMD, SMA and ALS).

Thus, there remains a need for methods for modulation of genes (including preferential modulation of aberrantly expressed genes and/or mutant alleles) involved in rare diseases, including for the prevention and/or treatment of rare diseases such as Angelman's Syndrome, FHMD, ALS, FTD and SMA.

### SUMMARY

Disclosed herein are methods and compositions for diagnosing, preventing and/or treating rare diseases such as Angelman's Syndrome, FHMD, ALS, FTD and SMA. In particular, provided herein are methods and compositions for modifying (e.g., modulating expression of) specific genes so as to treat these diseases including the use of engineered transcription factor repressors and nucleases.

Provided herein is a genetic modulator of a C9orf72 gene, the modulator comprising a DNA-binding domain (e.g., zinc finger protein (ZFP), a TAL-effector domain protein (TALE) or single guide RNA) that binds to a target site of at least 12 nucleotides in the C9orf72 gene; and a transcriptional regulatory domain (e.g., repression domain or activation domain) or nuclease domain. One or more polynucleotides (e.g., viral or nonviral gene delivery vehicle, for example an AAV vector) encoding one or more of the genetic modulators described herein are also provided. In other aspects, described herein are pharmaceutical compositions comprising one or more polynucleotides and/or or one or more gene delivery vehicles as provided herein. In aspects in which the genetic modulator comprises a nuclease domain, the genetic modulator (and pharmaceutical composition comprising the one or more genetic modulators or polynucleotides encoding the one or more genetic modulators) cleaves the C9orf72 gene, while in aspects wherein the genetic modulator comprises a regulator domain, the genetic modulator (and pharmaceutical composition comprising the one or more genetic modulators or polynucleotides encoding the one or more genetic modulators) modulates (for example represses or activates) the expression of the C9orf72 gene. Sense and/or antisense strands of the gene may be bound and/or modulated. The pharmaceutical composition comprising one or more nuclease genetic modulators may further comprise a donor molecule that is integrated into the cleaved C9orf72 gene. Also provided herein are isolated cells (including cell populations) comprising one or more genetic modulators; one or more polynucleotides; one or more gene delivery vehicles; and/or one or more pharmaceutical compositions as described herein. Methods and uses for modulating expressing (e.g., repressing) a C9orf72 gene in a cell (in vitro, in vivo or ex vivo) are also provided, the methods comprising administering (via any method including but not limited to intracerebroventricular, intrathecal, intracranial, retro-orbital (RO), intravenous or intracisternal) one or more genetic modulators; one or more polynucleotides; one or more gene delivery vehicles; and/or one or more pharmaceutical compositions as described herein to the cells. The methods can be used for the treatment and/or prevention of Amyotrophic Lateral Sclerosis (ALS) or Frontotemporal dementia (FTD) in a subject. Uses of one or more one or more genetic modulators; one or more polynucleotides; one or more gene delivery vehicles; and/or one or more pharmaceutical compositions for the treatment and/or prevention of Amyotrophic Lateral Sclerosis (ALS) or Frontotemporal dementia (FTD) in a subject are also provided. Also provided is a kit comprising one or more genetic modulators; one or more polynucleotides; one or more gene delivery vehicles; and/or one or more pharmaceutical compositions as described herein and, optionally, instructions for use.

Thus, in one aspect, engineered (non-naturally occurring) genetic modulators (e.g., repressors) of one or more genes are provided. These genetic modulators may comprise systems (e.g., zinc finger proteins, TAL effector (TALE) proteins or CRISPR/dCas-TF) that modulate (e.g., repress) expression of an allele. Expression of wild-type and/or mutant alleles may be modulated. In certain embodiments, the modulation of the mutant allele is at a greater level than the wild-type allele (e.g., wild-type allele is repressed no more than 50% of normal but a mutant allele is repressed by at least 70% as compared to untreated control). For example, in one embodiment, an engineered transcription factor can be used to repress the expression of the Ube3a-ATS RNA for the treatment of Angelman Syndrome. In FSHD1, a mutation that leads to the expression of DUX4 in somatic tissues (normally epigenetically silenced after germline development, see van der Maarel et al (2011) Trends Mol Med. 17(5):252-8. doi: 10.1016/j.molmed.2011.01.001). Thus, in some embodiments, engineered transcription factors can be used to repress its expression for the treatment of FSHD1. Similarly, an expansion mutation in a C9orf72 allele leads to expression of both a sense and anti-sense RNA product associated with ALS and FTD, so in one embodiment, provided are engineered transcription factors designed to repress expression of these mutant C9orf72 alleles for the treatment of ALS or FTD. In some embodiments, transcription factors engineered to induce the expression of the SMN1 and/or SMN2 genes for the treatment of SMA or to induce the expression of the paternal allele of UBE34 for the treatment of AS are provided. Engineered zinc finger proteins or TALEs are non-naturally occurring zinc finger or TALE proteins whose DNA binding domains (e.g., recognition helices or RVDs) have been altered (e.g., by selection and/or rational design) to bind to a pre-selected target site. Any of the zinc finger proteins described herein may include 1, 2, 3, 4, 5, 6 or more zinc fingers, each zinc finger having a recognition helix that binds to a target subsite in the selected sequence(s) (e.g., gene(s)). In certain embodiments, the ZFP-TFs comprise a ZFP having the recognition helix regions as shown in a single row of Table 1. Similarly, any of the TALE proteins described herein may include any number of TALE RVDs. In some embodiments, at least one RVD has non-specific DNA binding. In some embodiments, at least one recognition helix (or RVD) is non-naturally occurring. In certain embodiments, the TALE-TF comprises a TALE that binds to at least 12 base pairs of a target site as shown in Table 1. A CRISPR/Cas-TF includes a single guide RNA that binds to a target sequence. In certain embodiments, the engineered transcription factor binds to (e.g., via a ZFP, TALE or sgRNA DNA binding domain) to an at least 9-12 base pair target site in a disease associated gene, for example a target site comprising at least 9-20 base pairs (e.g., 9, 10, 11, 12, 13, 14, 15, 16, 17, 18, 19, 20 or more), including contiguous or non-contiguous sequences within these target sites (e.g., a target site as shown in Table 1). In certain embodiments, the genetic modulator comprises a DNA-binding molecule (ZFP, TALE, single guide RNA) as described herein operably linked to a transcriptional repression domain (to form a genetic repressor) or transcriptional activation domain (to form a genetic repressor). In other embodiments, the genetic repressor (e.g., that represses expression of the gene via modification of the sequence) comprises a DNA-binding molecule (ZFP, TALE, single guide RNA) as described herein operably linked to at least one nuclease domain (e.g., one, two or more nuclease domains). The resulting artificial nuclease is capable of genetically modifying (by insertions and/or deletions) the target gene, for example, within the DNA-binding domain target sequence(s); within the cleavage site(s); near (1-50 or more base pairs) from the target sequence(s) and/or cleavage site(s); and/or between paired target sites when a pair of nucleases is used for cleavage such that expression of the gene is repressed (inactivated).

Thus, the zinc finger proteins (ZFPs), Cas protein of a CRISPR/Cas system or TALE proteins as described herein can be placed in operative linkage with a regulatory domain (or functional domain) as part of a fusion molecule. The functional domain can be, for example, a transcriptional activation domain, a transcriptional repression domain and/or a nuclease (cleavage) domain. By selecting either an activation domain or repression domain for use with the DNA-binding molecule, such molecules can be used either to activate or to repress gene expression. In certain embodiments, the functional or regulatory domains can play a role in histone post-translational modifications. In some instances, the domain is a histone acetyltransferase (HAT), a histone deacetylase (HDAC), a histone methylase, or an enzyme that sumolyates or biotinylates a histone or other enzyme domain that allows post-translation histone modification regulated gene repression (Kousarides (2007) Cell 128:693-705). In some embodiments, a molecule comprising a ZFP, dCas or TALE targeted to a gene (e.g. C9orf72, Ube3a-ATS, DUX4) as described herein fused to a transcriptional repression domain that can be used to down-regulate gene expression is provided. In other embodiments, a molecule comprising a ZFP, dCAS or TALE targeted to a gene (e.g., C9orf72, UBE34, SMN1 or SMN2) to activate gene expression is provided. In some embodiments, the methods and compositions of the invention are useful for treating eukaryotes. In certain embodiments, the activity of the regulatory domain is regulated by an exogenous small molecule or ligand such that interaction with the cell's transcription machinery will not take place in the absence of the exogenous ligand. Such external ligands control the degree of interaction of the ZFP-TF, CRISPR/Cas-TF or TALE-TF with the transcription machinery. The regulatory domain(s) may be operatively linked to any portion(s) of one or more of the ZFPs, dCas or TALEs, including between one or more ZFPs, dCas or TALEs, exterior to one or more ZFPs, dCas or TALEs and any combination thereof. In preferred embodiments, the regulatory domain results in a repression of gene expression of the targeted gene (e.g., C9orf72, Ube3a-ATS, DUX4). In other preferred embodiments, the regulatory domain results in a activation of gene expression of the targeted gene (e.g., C9orf72, UBE34, SMN1 and/or SMN2). Any of the fusion proteins described herein may be formulated into a pharmaceutical composition.

In some embodiments, the methods and compositions of the invention include use of two or more fusion molecules as described herein, for instance two or more C9orf72, Ube3a-ATS and/or DUX4 modulators (artificial transcription factors and/or artificial nucleases). The two or more fusion molecules may bind to different target sites and comprise the same or different functional domains. Alternatively, the two or more fusion molecules as described herein may bind to the same target site but include different functional domains. In some instances, three or more fusion molecules are used, in others, four or more fusion molecules are used, while in others, 5 or more fusion molecules are used. In preferred embodiments, the two or more, three or more, four or more, or five or more fusion molecules (or components thereof) are delivered to the cell as nucleic acids. In preferred embodiments, the fusion molecules cause a repression of the expression of the targeted gene. In some embodiments, two fusion molecules are given at doses where each molecule is active on its own but in combination the repression activity is additive. In preferred embodiments, two fusion molecules are given at doses where neither is active on its own, but in combination, the repression activity is synergistic.

In some embodiments, the engineered DNA binding domains as described herein can be placed in operative linkage with nuclease (cleavage) domains as part of a fusion molecule. In some embodiments, the nuclease comprises a Ttago nuclease. In other embodiments, nuclease systems such as the CRISPR/Cas system may be utilized with a specific single guide RNA to target the nuclease to a target location in the DNA. In certain embodiments, pharmaceutical compositions comprising the modified stem, muscle, and/or neuronal cells are provided.

In yet another aspect, a polynucleotide encoding any of the DNA binding domains described herein is provided.

In other aspects, the invention comprises delivery of a donor nucleic acid to a target cell. The donor may be delivered prior to, after, or along with the nucleic acid encoding the nuclease(s). The donor nucleic acid may comprise an exogenous sequence (transgene) to be integrated into the genome of the cell, for example, an endogenous locus. In some embodiments, the donor may comprise a full-length gene or fragment thereof flanked by regions of homology with the targeted cleavage site. In some embodiments, the donor lacks homologous regions and is integrated into a target locus through homology independent mechanism (i.e. NHEJ). The donor may comprise any nucleic acid sequence, for example a nucleic acid that, when used as a substrate for homology-directed repair of the nuclease-induced double-strand break, leads to a donor-specified deletion to be generated at the endogenous chromosomal locus or, alternatively (or in addition to), novel allelic forms of (e.g., point mutations that ablate a transcription factor binding site) the endogenous locus to be created. In some aspects, the donor nucleic acid is an oligonucleotide wherein integration leads to a gene correction event, or a targeted deletion. In some embodiments, the donor encodes a transcription factor capable of repressing target gene expression. In other embodiments, the donor encodes an RNA molecule that inhibits expression of the targeted protein.

In some embodiments, the polynucleotide encoding the DNA binding protein is an mRNA. In some aspects, the mRNA may be chemically modified (See e.g. Kormann et al, (2011) Nature Biotechnology 29(2):154-157). In other aspects, the mRNA may comprise an ARCA cap (see U.S. Pat. Nos. 7,074,596 and 8,153,773). In further embodiments, the mRNA may comprise a mixture of unmodified and modified nucleotides (see U.S. Patent Publication 2012-0195936).

In yet another aspect, a gene delivery vector comprising any of the polynucleotides (e.g., repressors) as described herein is provided. In certain embodiments, the vector is an adenovirus vector (e.g., an Ad5/F35 vector), a lentiviral vector (LV) including integration competent or integration-defective lentiviral vectors, or an adenovirus associated viral vector (AAV). In certain embodiments, the AAV vector is an AAV2, AAV6, AAV8 or AAV9 vector or pseudotyped AAV vector such as AAV2/8, AAV2/5, AAV2/9 and AAV2/6. In some embodiments, the AAV vector is an AAV vector capable of crossing the blood-brain barrier (e.g. U.S. 20150079038). In other embodiments, the AAV is a self-complementary AAV (sc-AAV) or single stranded (ss-AAV) molecule. Also provided herein are adenovirus (Ad) vectors, LV or adenovirus associate viral vectors (AAV) comprising a sequence encoding at least one nuclease (ZFN or TALEN) and/or a donor sequence for targeted integration into a target gene. In certain embodiments, the Ad vector is a chimeric Ad vector, for example an Ad5/F35 vector. In certain embodiments, the lentiviral vector is an integrase-defective lentiviral vector (IDLV) or an integration competent lentiviral vector. In certain embodiments, the vector is pseudo-typed with a VSV-G envelope, or with other envelopes.

Additionally, pharmaceutical compositions comprising the nucleic acids, and/or fusions such as artificial transcription factors or nucleases (e.g., ZFPs, Cas or TALEs or fusion molecules comprising the ZFPs, Cas or TALEs) are also provided. For example, certain compositions include a nucleic acid comprising a sequence that encodes one of the ZFPs, Cas or TALEs described herein operably linked to a regulatory sequence, combined with a pharmaceutically acceptable carrier or diluent, wherein the regulatory sequence allows for expression of the nucleic acid in a cell. In certain embodiments, the ZFPs, Cas, CRISPR/Cas or TALEs encoded modulate a wild-type and/or mutant allele. In some embodiments, the mutant allele is preferentially modulated, e.g., is repressed or activated more than the wild-type allele. In some embodiments, pharmaceutical compositions comprise ZFPs, CRISPR/Cas or TALEs that preferentially modulate a mutant allele and ZFPs, CRISPR/Cas or TALEs that modulate a neurotrophic factor. Protein based compositions include one of more ZFPs, CRISPR/Cas or TALEs as disclosed herein and a pharmaceutically acceptable carrier or diluent.

In yet another aspect also provided is an isolated cell comprising any of the proteins, fusion molecules, polynucleotides and/or compositions as described herein. The isolated cell may be used for non-therapeutic uses such as the provision of cell or animal models for diagnostic and/or screening methods and/or for therapeutic uses such as ex vivo cell therapy.

In yet another aspect, also provided are pharmaceutical compositions comprising one or more genetic modulators, one or more polynucleotides (e.g., gene delivery vehicles) and/or one or more (e.g., a population of) isolated cells as described herein. In certain embodiments, the pharmaceutical composition comprises two or more genetic modulators. For example, certain compositions include a nucleic acid comprising a sequence that encodes one or more genetic modulators of one of genes associated with the rare disease (e.g., C9orf72, Ube3a-ATS, DUX4) as described herein. In certain embodiments, the genetic modulator(s) (e.g., comprising ZFPs, Cas or TALEs described herein) are operably linked to a regulatory sequence, combined with a pharmaceutically acceptable carrier or diluent, where the regulatory sequence allows for expression of the nucleic acid in a cell. In certain embodiments, the ZFPs, CRISPR/Cas or TALEs encoded are specific for a mutant or wild type allele (e.g., C9orf72). In some embodiments, pharmaceutical compositions comprise ZFP-TFs, CRISPR/Cas-TFs or TALE-TFs that modulate a mutant and/or wild type allele (e.g., C9orf72), including TFs that preferentially modulate (activate or repress at greater levels) the mutant allele as compared to the wild-type allele. Protein-based compositions include one of more genetic modulators as disclosed herein and a pharmaceutically acceptable carrier or diluent.

The invention also provides methods and uses for repressing gene expression in a subject in need thereof (e.g., a subject with a rare disease as described herein), including by providing to the subject one or more polynucleotides, one or more gene delivery vehicles, and/or a pharmaceutical composition as described herein. In certain embodiments, the compositions described herein are used to repress mutant C9orf72 expression in the subject, including for treatment and/or prevention of ALS or FTD. The compositions described herein repress gene expression for sustained periods of time (4 weeks, 3 months, 6 months to year or more) in the brain (including but not limited to the frontal cortical lobe including but not limited to the prefrontal cortex, parietal cortical lobe, occipital cortical lobe, temporal cortical lobe including by not limited to the entorhinal cortex, hippocampus, brain stem, striatum, thalamus, midbrain, cerebellum) and spinal cord (including but not limited to lumbar, thoracic and cervical regions). The compositions described herein may be provided to the subject by any administration means, including but not limited to, intracerebroventricular, intrathecal, intracranial, intravenous, orbital (retro-orbital (RO)), intranasal and/or intracisternal administration. Kits comprising one or more of the compositions (e.g., genetic modulators, polynucleotides, pharmaceutical compositions and/or cells) as described herein as well as instructions for use of these compositions are also provided.

In another aspect, provided herein are methods for treating and/or preventing a CNS (e.g. AS, ALS, FTD and/or SMA) or muscle disorder (e.g. FSHD) using the methods and compositions described herein. In some embodiments, the methods involve compositions where the polynucleotides and/or proteins may be delivered using a viral vector, a non-viral vector (e.g., plasmid) and/or combinations thereof. In some embodiments, the methods involve compositions comprising stem cell populations comprising an artificial transcription factor or artificial nuclease (e.g., ZFP-TF, TALE-TF, Cas-TF, ZFN, TALEN, Ttago) or the CRISPR/Cas nuclease system of the invention. Administration of compositions as described herein (proteins, polynucleotides, cells and/or pharmaceutical compositions comprising these proteins, polynucleotides and/or cells) result in a therapeutic (clinical) effect, including, but not limited to, amelioration or elimination of any the clinical symptoms associate with AS, FSHD, ALS, FTD and/or SMA, as well as an increase in function and/or number of CNS cells (e.g., neurons, astrocytes, myelin, etc.) or muscle cells. In certain embodiments, the compositions and methods described herein reduce expression of their target gene (e.g., C9orf72), as compared to controls not receiving the artificial repressors as described herein, by at least 30%, or 40%, preferably by at least 50%, even more preferably by at least 70%, or at least 80% or at least 90%, or at least 95% or greater that 95%. In some embodiments, at least 50% reduction is achieved. In certain embodiments, the artificial repressor preferentially represses a mutant allele (for example, an expanded allele) as compared to a wild-type allele, for example by at least 20% (e.g., represses the wild-type allele no more than 50% and the mutant allele by at least 70%).

In a still further aspect, described here is a method of delivering a gene repressor to the brain of the subject using a viral or non-viral vector. In certain embodiments, the viral vector is an AAV9 vector. Delivery may be to any brain region, for example, the hippocampus or entorhinal cortex by any suitable means including via the use of a cannula. Any AAV vector that provides widespread delivery of the genetic modulator (e.g., repressor) to brain of the subject, including via anterograde and retrograde axonal transport to brain regions not directly administered the vector (e.g., delivery to the putamen results in delivery to other structures such as the cortex, substantia nigra, thalamus, etc.). In certain embodiments, the subject is a human and in other embodiments, the subject is a non-human primate. The administration may be in a single dose, or in a series of doses given at the same time, or in multiple administrations (at any timing between administrations).

Thus, in other aspects, described herein is a method of preventing and/or treating a disease (e.g., AS, FSHD, ALS, FTD and/or SMA) in a subject, the method comprising administering a repressor of a gene to the subject using AAV. In certain embodiments, the repressor is administered to the CNS (e.g., hippocampus and/or entorhinal cortex) or PNS (e.g., spinal cord/fluid) of the subject. In other embodiments, the repressor is administered intravenously. In certain embodiments, described herein is a method of preventing and/or treating ALS or FTD in a subject, the method comprising administering a repressor of a C9orf72 allele (wild-type and/or mutant) to the subject using one or more AAV vectors. In certain embodiments, the AAV encoding the genetic modulator is administered to the CNS (brain and/or CSF) via any delivery method including but not limited to, intracerebroventricular, intrathecal, intracranial, intravenous, intranasal, retro-orbital, or intracisternal delivery. In other embodiments, the AAV encoding the repressor is administered directly into the parenchyma (e.g., hippocampus and/or entorhinal cortex) of the subject. In other embodiments, the AAV encoding the repressor is administered intravenously (IV). In any of the methods described herein, the administering may be done once (single administration) or may be done multiple times (with any time between administrations) at the same or different doses per administration. When administered multiple times, the same or different dosages and/or delivery vehicles of modes of administration may be used (e.g., different AAV vectors administered IV and/or ICV). The methods include methods of reducing the loss of muscle function, the loss of physical coordination, stiffening of muscles, muscle spasms, loss of speech functions, difficulty of swallowing, cognitive impairment, method of reducing loss of motor function, and/or methods of reducing loss of one or more cognitive functions in ALS subjects, all in comparison with a subject not receiving the method, or in comparison to the subject themselves prior to receiving the methods. Thus, the methods described herein result in reduction in biomarkers and/or symptoms of rare diseases such as ALS or FTD, including one or more the following: the loss of muscle function, the loss of physical coordination, stiffening of muscles, muscle spasms, loss of speech functions, difficulty of swallowing, cognitive impairment, changes in blood and/or cerebral spinal fluid chemistries associated with ALS, including G-CSF, IL-2, IL-15, IL-17, MCP-1, MIP-1α, TNF-α, and VEGF levels (see Chen et al (2018) Front Immunol. 9:2122. doi: 10.3389/fimmu.2018.02122), a reduction in decreases in cortical thickness of atlas-based dorsal and ventral subdivisions of the precentral and postcentral cortex, ALSFRS-R, and MUNIX for the musculus abductor digiti minimi (see Wirth et al (2018) Front Neurol. 9:614. doi: 10.3389/fneur.2018.00614) and/or other biomarkers known in the art. In certain embodiments, the methods may further comprise administering one or more genetic repressors of tau (MAPT), for example in subjects with FTD. See, e.g., U.S. Publication No. 20180153921.

In any of the methods described herein, the repressor of the targeted allele may be a ZFP-TF, for example a fusion protein comprising a ZFP that binds specifically to an allele and a transcriptional repression domain (e.g., KOX, KRAB, etc.). In other embodiments, the repressor of the targeted allele may be a TALE-TF, for example a fusion protein comprising a TALE polypeptide that binds specifically to a gene allele and a transcriptional repression domain (e.g., KOX, KRAB, etc.). In some embodiments, the targeted allele repressor is a CRISPR/Cas-TF where the nuclease domains in the Cas protein have been inactivated such that the protein no longer cleaves DNA. The resultant Cas RNA-guided DNA binding domain is fused to a transcription repressor (e.g. KOX, KRAB etc.) to repress the targeted allele. In some embodiments, the engineered transcription factor is able to repress expression of a mutated allele but not the wild type allele. In further embodiments, the DNA binding molecule preferentially recognizes a hexameric GGGGCC expansion.

In some embodiments, the sequence encoding a genetic repressor as described herein (e.g., ZFP-TF, TALE-TF or CRISPR/Cas-TF) is inserted (integrated) into the genome while in other embodiments the sequence encoding the repressor is maintained episomally. In some instances, the nucleic acid encoding the TF fusion is inserted (e.g., via nuclease-mediated integration) at a safe harbor site comprising a promoter such that the endogenous promoter drives expression. In other embodiments, the repressor (TF) donor sequence is inserted (via nuclease-mediated integration) into a safe harbor site and the donor sequence comprises a promoter that drives expression of the repressor. In some embodiments, the promoter sequence is broadly expressed while in other embodiments, the promoter is tissue or cell/type specific. In preferred embodiments, the promoter sequence is specific for neuronal cells. In other preferred embodiments, the promoter sequence is specific for muscle cells. In especially preferred embodiments, the promoter chosen is characterized in that it has low expression. Non-limiting examples of preferred promoters include the neural specific promoters NSE, Synapsin, CAMKiia and MECPs. Non-limiting examples of ubiquitous promoters include CMV, CAG and Ubc. Further embodiments include the use of self-regulating promoters as described in U.S. Patent Publication No. 2015/0267205. Further embodiments include the use of self-regulating promoters as described in US Publication No. 20150267205.

In any of the methods described herein, the method can yield about 50% or greater, 55% or greater, 60% or greater, 65% or greater, about 70% or greater, about 75% or greater, about 85% or greater, about 90% or greater, about 92% or greater, or about 95% or greater repression, 98% or greater, or 99% or greater of the target alleles (e.g., mutant or wild-type C9orf72) in one or more neurons of a subject (e.g., a subject with ALS). In certain embodiments, expression of the wild-type allele is repressed no more than 50% in the subject (as compared to untreated subjects) while the mutant allele is repressed at least 70% (70% or any value thereabove) in the subject (as compared to untreated subjects).

In still further embodiments, the repressor may comprise a nuclease (e.g., ZFN, TALEN and/or CRISPR/Cas system) that represses the targeted allele by cleaving and thereby inactivating the targeted allele. In certain embodiments, the nuclease introduces an insertion and/or deletion (“indel”) via non-homologous end joining (NHEJ) following cleavage by the nuclease. In other embodiments, the nuclease introduces a donor sequence (by homology or non-homology directed methods), in which the donor integration inactivates the targeted allele. In some embodiments, the targeted gene is a wild-type or mutant C9orf72, Ube32-ATS and/or DUX4 gene comprising a target site of 9-20 more nucleotides to which the DNA-binding domain binds.

In any of the methods described herein, the regulator (e.g. nuclease, repressor or activator) may be delivered to the subject (e.g., brain or muscle) as a protein, polynucleotide or any combination of protein and polynucleotide. In certain embodiments, the repressor(s) is (are) delivered using an AAV vector. In other embodiments, at least one component of the regulator (e.g., sgRNA of a CRISPR/Cas system) is delivered as an RNA form. In other embodiments, the regulator(s) is (are) delivered using a combination of any of the expression constructs described herein, for example one repressor (or portion thereof) on one expression construct (AAV9) and one repressor (or portion thereof) on a separate expression construct (AAV or other viral or non-viral construct).

Furthermore, in any of the methods described herein, the regulator (e.g., repressor) can be delivered to a cell (ex vivo or in vivo) at any concentration (dose) that provides the desired effect. In preferred embodiments, the regulator is delivered using an adeno-associated virus (AAV) vector at 10,000-500,000 vector genome/cell (or any value therebetween). In certain embodiments, the regulator is delivered using a lentiviral vector at MOI between 250 and 1,000 (or any value therebetween). In other embodiments, the regulator is delivered using a plasmid vector at 0.01-1,000 ng/100,000 cells (or any value therebetween). In other embodiments, the repressor is delivered as mRNA at 150-1,500 ng/100,000 cells (or any value therebetween). Furthermore, for in vivo uses, in any of the methods described herein, the genetic modulator(s) (e.g., repressors) can be delivered at any concentration (dose) that provides the desired effect in a subject in need thereof. In preferred embodiments, the repressor is delivered using an adeno-associated virus (AAV) vector at 10,000-500,000 vector genome/cell (or any value therebetween). In certain embodiments, the repressor is delivered using a lentiviral vector at MOI between 250 and 1,000 (or any value therebetween). In other embodiments, the repressor is delivered using a plasmid vector at 0.01-1,000 ng/100,000 cells (or any value therebetween). In other embodiments, the repressor is delivered as mRNA at 0.01-3000 ng/number of cells (e.g., 50,000-200,000 (e.g., 100,000) cells (or any value therebetween). In other embodiments, the repressor is delivered using an adeno-associated virus (AAV) vector at a fixed volume of 1-300 ul to the brain parenchyma at 1E11-1E14 VG/ml. In other embodiments, the repressor is delivered using an adeno-associated virus (AAV) vector at a fixed volume of 0.5-10 ml to the CSF at 1E11-1E14 VG/ml.

In any of the methods described herein, the method can yield about 50% or greater, 55% or greater, 60% or greater, 65% or greater, about 70% or greater, about 75% or greater, about 85% or greater, about 90% or greater, about 92% or greater, or about 95% or greater modulation (e.g., repression) of the targeted allele(s) in one or more cells of the subject. In some embodiments, wild-type and mutant alleles are modulated differently, for example the mutant allele is preferentially modified as compared to the wild-type allele (e.g., mutant allele repressed by at least 70% and the wild-type allele is repressed by no more than 50%).

In further aspects, the transcription factors as described herein, such as a transcription factors comprising one or more of a zinc finger protein (ZFP TFs), a TALEs (TALE-TF), and a CRISPR/Cas-TFs for example, ZFP-TFs, TALE-TFs or CRISPR/Cas-TFs, are used to repress expression of a mutant and/or wild type allele in of the brain (e.g., neuron), or in a muscle cell, of a subject. The repression can be about 50% or greater, 55% or greater, 60% or greater, 65% or greater, 70% or greater, about 75% or greater, about 85% or greater, about 90% or greater, about 92% or greater, or about 95% or greater repression of the targeted alleles in the one or more cells of the subject as compared to untreated (wild-type) cells of the subject. In certain embodiments, repression of the wild-type allele is not more than 50% (as compared to untreated cells or subjects) and repression of the mutant (diseased or isoform variant) is at least 70% (as compared to untreated cells or subjects). In certain embodiments, the targeted-modulating transcription factor can be used to achieve one or more of the methods described herein.

Thus, described herein are methods and compositions for modulating expression of genes associated with the rare disorders disclosed herein, including repression with or without expression of an exogenous sequence (such as an artificial TF). The compositions and methods can be for use in vitro (e.g., for the provision of cells for the study of the target gene via its modulation; for drug discovery; and/or to make transgenic animals and animal models), in vivo or ex vivo, and comprise administering an artificial transcription factor or nuclease that includes a DNA-binding molecule targeted to the gene associated with the rare disease, optionally in the case of a nuclease with a donor that is integrated into the gene following cleavage by the nuclease. In some embodiments, the donor gene (transgene) is maintained extrachromosomally in a cell. In certain embodiments, the cell is in a patient with the disease. In other embodiments, the cell is modified by any of the methods described herein, and the modified cell is administered to a subject in need thereof (e.g., a subject with the rare disease). Genetically modified cells (e.g., stem cells, precursor cells, T cells, muscle cells, etc.) comprising a genetically modified gene (e.g., an exogenous sequence) are also provided, including cells made by the methods described herein. These cells can be used to provide therapeutic protein(s) to a subject with the rare disease, for example by administering the cell(s) to a subject in need thereof or, alternatively, by isolating the protein produced by the cell and administering the protein to the subject in need thereof (enzyme replacement therapy).

Also provided is a kit comprising one or more of the genetic modulators (e.g., repressors) and/or polynucleotides comprising components of and/or encoding the target-modulators (or components thereof) as described herein. The kits may further comprise cells (e.g., neurons or muscle cells), reagents (e.g., for detecting and/or quantifying a protein, for example in CSF) and/or instructions for use, including the methods as described herein.

### BRIEF DESCRIPTION OF THE DRAWINGS

FIGS. 1A and 1B are schematics of the human chromosome 15q11-13 region, and shows differences in the maternal (FIG. 1B) and paternal (FIG. 1A) alleles. Paternally expressed genes are shown as grey boxes and maternally expressed genes are shown as black boxes. Biallelically genes are shown as dark grey boxes. Right arrow indicates gene transcription on “+” strand, whereas left arrow indicates gene transcription on the “−” strand. AS-IC (triangle) and PWS-IC (ellipse) are shaded depending on histone modification in the area. AS-IC is dormant (gray triangle) on the paternal chromosome, whereas, on the maternal chromosome, it is acetylated and methylated at H3-lys4 (triangle), thus active. PWS-IC is active on the paternal chromosome (upper ellipse), since it is also acetylated and methylated at H3-lys4. However, PWS-IC at the maternal chromosome is methylated at H3-lys9 and repressed (lower ellipse). Differentially, the CpG methylated region (differentially methylated region 1 [DMR1]) in small nuclear ribonucleoprotein polypeptide N (SNRPN) exon 1 partially overlaps with PWS-IC. Note that DMR1 on the maternal but not paternal chromosome is methylated (black pin). Ubiquitin protein ligase E3A antisense transcript (UBE3A-ATS) originating upstream of SNRPN can either be a degradable complex with UBE3A transcript or prevent the extension of the ubiquitin protein ligase E3A (UBE3A) transcript (collision or upstream histone modifications represented by “X”).

FIGS. 2A through 2D show repression of C9orf72 expression “Total C9” in the indicated cell types using the indicated artificial transcription factors (ZFP-TFs). In addition, the figures show repression of the expression of a longer mRNA isoform comprising intron 1A, which is predominantly, although not exclusively produced by the expanded, mutant allele: “Isoform specific”. FIG. 2A depicts the PCR assays used for the Total C9 assay and the Isoform specific assay. The top of the figure depicts the genomic sequences of the wildtype and expanded alleles, while the bottom of the figure shows the mRNA products made from each allele. Arrow sets on the mRNA drawings depict the PCR targets used in the Total C9 assay and the Isoform specific assay. FIGS. 2B through 2D show the results of the assays for different exemplary ZFP-TFs in graphs depicting Total C9orf72 expression in a wild-type cell line in a 3^{rd} round of screening (“Round 3”); the graphs second from the left show Total C9orf72 expression in “C9” cell line (defined as “5/>145”; referring to the number of G4C2 repeats on the wildtype allele, (5)/compared to the G4C2 repeats on the expanded allele, >145) in a 3^{rd} round of screening (“Round 3”); the graphs second from the right show Total C9orf72 expression in C9 cell line as defined above in a 2^{nd} round of screening (“Round 2”); and the right most graphs show the results from the Isoform-specific C9orf72 assay (see Example 2). In Round 2 screen was done in C9 line from patients evaluating isoform (or disease) specific C9 vs. total C9 levels following ZFP treatment. In Round 3, total C9 was evaluated in C9 line from patients compare to wild type (WT) lines from a health individual in order to evaluate ZFP effects on C9 WT allele. For each ZFP, concentrations of 1, 3, 10, 30, 100 and 300 ng mRNA are shown from left to right (see Example 2 for details). FIG. 2B shows results for ZFP-TFs comprising ZFPs designated 74949, 74951, 74954, 74955 and 74964 in the top graphs and 74969, 74971, 74973, 74978 and 74979 in the bottom graphs. FIG. 2C shows results for ZFP-TFs comprising ZFPs designated 74983, 74984, 74986, 74987 and 74988 in the top graphs and 74997, 74998, 75001 and 75003 in the bottom graphs. FIG. 2D shows results for ZFP-TFs comprising ZFPs designated 75023, 75027, 75031, 75032, 75055 and 75078 in the top graphs and 75090, 75105, 75109, 75114 and 75115 in the bottom graphs. The sequence at the bottom of graphs represents the DNA binding motif for that ZFP. Each ZFP will bind to three hexanucleotide repeat contain that motif.

FIG. 3 shows results of microarray analysis results showing specificity of the indicated repressors (75027 and 75115) for the C9orf72 gene. Analysis was performed 24 hours after administration to C9021 cells of the repressors in mRNA form at 300 ng. The left plot shows results using ZFP repressor 75027 and the right plot shows results using ZFP repressor 75115. Results are also discussed in Example 3.

### DETAILED DESCRIPTION

Disclosed herein are compositions and methods for the prevention and/or treatment of the rare diseases Angelman's Syndrome, FHMD, ALS and/or SMA. In particular, the compositions and methods described herein are used to repress the expression of a disease associated gene to prevent or treat these diseases.

Angelman's Syndrome (AS) is a neurodevelopmental disorder with a prevalence of between 1/10,000 and 1/20,000 individuals. Characterized by intellectual disability, lack of speech, jerky movements, sleep disorders and seizures, AS patients also display a happy demeanor, laughing frequently while being drawn to water. Developmental delays are evident in these patients within the first year of life and typically they reach a developmental plateau between 24 and 30 months of life. In addition, seizures in 80% AS patients exhibit a characteristic EEG signature that can be used to confirm diagnosis, where seizure onset occurs around three years of life and continues into adulthood (Clayton-Smith (2003) J Med Genet 40(2): 87-95). Life expectancy for AS patients is nearly normal although drownings occur with some frequency in younger patients (see Bird (2014) Appl Clin Gene (7):93-104).

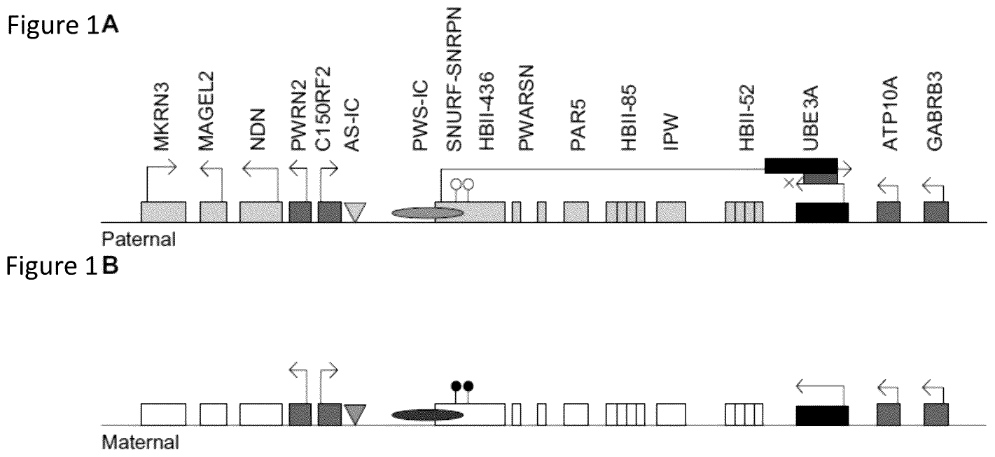

AS is associated with deficient expression of the UBE3A gene which encodes E6 associated protein (an E3 ubiquitin ligase). E6 associated protein is involved in the ubiquination of proteins bound for destruction, so the phenotypic characteristics of the disease may involve accumulation of these substrates. The UBE3A gene is located in the 15q11-13 interval on chromosome 15 (see FIG. 1, adapted from Bird, ibid). This locus is subject to genetic imprinting which is a type of epigenetic regulation leading to preferential expression a gene from the paternal or maternal allele. Imprinting occurs in gametogenesis where some regions of the DNA are differentially methylated depending on whether the gamete is male or female. In oocytes, hypermethylated CpG islands are associated with active transcription regions, while in the male germline, methylation is not as concentrated in imprinted genes, and the promoters of these genes that will bear a paternal imprint are less CpG rich in comparison with maternally imprinted genes (Stewart et al (2016) Epigenomics 8(10):1399-1413). UBE3A is a gene that is expressed biallelically throughout the body except for some specific cells of the brain. In neurons in both the developing and adult brain, UBE3A is expressed from the maternal allele only where the promoter on the maternal allele is heavily methylated. Thus, if there is a mutation in this region in the maternal allele, the paternal allele is not able to compensate. In AS patients with a molecular diagnosis, approximately 78.2% of patients have some type of deletion encompassing the maternal UBE3A gene, 11.2% have specific mutations within the UBE3A gene itself, and 7.7% have mutations associated with faulty genetic imprinting, (Bird, ibid).

To ensure the silencing of the paternal UBE3A allele in neurons, there is a long antisense RNA that is produced on the paternal allele (see FIG. 1) known as Ube3a-ATS. This antisense RNA is an atypical RNA polymerase II transcript from a paternally imprinted locus that appears to suppress paternal UBE3A expression in cis. The promoter for Ube3a-ATS appears to be at and upstream of the center for DNA methylation known as the Prader-Willi syndrome (PWS)/Angelman syndrome (AS) region imprinting center (also known as the PWS IC), and it has been shown that deletion of the PWS IC in mice represses the expression of Ube3a-ATS, and relieves the repression of the paternal UBE3A allele (Meng et al (2012) Hum Mol Genet 21(13):3001-3012). In addition, Bailus et al (2016, Mol Ther 24(3):548-55) showed that use of an artificial zinc finger transcription factor directed to the paternal UBE34 promoter caused wide spread expression of UBE3A in the brain in a mouse model of AS.

Currently there is no cure for AS, and treatment of these patients focuses on support therapies and approaches to mitigate the symptoms of the disease. Thus, described herein are compositions and methods for upregulating paternal UBE3A expression (e.g., using an artificial transcription factor as described herein that binds to a target site of at least 9-20 nucleotides in the target allele) and/or by inserting a donor into a cell of the subject, which donor encodes a wild-type (functional) UBE3A. Thus, activating paternal UBE3A can be used to treat and/or prevent AS.

Alternatively, or in addition to activation of paternal UBE3A expression, the compositions and methods described herein can also be used to suppress the expression of the Ube3a-ATS RNA to provide a treatment for this disease. Similarly, the use of one or more engineered nucleases can be used to knock out the Ube3a-ATS coding sequence and/or promoter, thereby treating and/or preventing AS and its symptoms.

Facioscapulohumeral Muscular Dystrophy (FSHD), as with most muscular dystrophies, is a neuromuscular disease, named for the regions of the body most noticeably affected, the face (facio), shoulder blades (scapula) and upper arms (humeral). It is the third most common myopathy after Duchenne's and Becker Muscular Dystrophies. Weakness involving the facial muscles or shoulders is usually the first symptom of this disease. Facial muscle weakness often makes it difficult to drink from a straw, whistle, or turn up the corners of the mouth when smiling. Weakness in muscles around the eyes can prevent the eyes from closing fully while a person is asleep, which can lead to dry eyes and other eye problems. The signs and symptoms of FSHD usually appear in adolescence. However, the onset and severity of the condition varies widely and can also be displayed asymmetrically (Bao et al (2016) Intractable Rare Dis Res 5(3): 168-176). Milder cases may not become noticeable until later in life, whereas rare severe cases become apparent in infancy or early childhood. The disease is an autosomal dominant one, with prevalence ranging from 1/8300 to 1/20,000 (Ansseau et al (2017) Genes 8(3): p. 93).

Recent studies have primarily attributed pathogenesis of FSHD to the aberrant expression of a normally dormant gene, DUX4. DUX4 is a double homeodomain transcription factor (double homeobox protein, 4) encoded within the D4Z4 tandem repeat. In a healthy individual, the subtelomeric region of chromosome 4q contains 11-100 copies of the 3.3 kb D4Z4 macrosatellite repeat, each with a copy of DUX4. However, DUX4 is not expressed in normal functioning somatic tissues such as well-differentiated muscles fibers. While DUX4 is expressed in early development, it is transcriptionally silenced during cellular differentiation of somatic tissues by CpG methylation of D4Z4 repeats. The gene encodes a transcription factor that may be involved in the activation of a transcription pathway in stem cells.

The D4Z4 array is a region of repeated tandem 3.3-kb repeat units on chromosome 4. These arrays are in sub-telomeric regions of 4q and 10q and have 1-100 repeat units. FSHD is associated with an array of 1-10 units at 4q35. The majority of FSHD patients with <11 repeat units in the D4Z4 array will experience onset of symptoms with about 95% penetrance by 20 years of age. There is no treatment that can halt or reverse the effects of FSHD although there are medications (e.g. NSAIDs) and procedures (e.g., shoulder surgery to stabilize the shoulder blades) that can alleviate the symptoms.

There are two types of FSHD: FSHD type 1 (FSDH1) and FSHD type 2 (FSHD2), with 95% of cases being FSHD1. FSHD1 is caused by a contraction of the polymorphic D4Z4 macrosatellite repeat array in chromosome 4. The D4Z4 macrosatellite repeat consists of a 3.3 kb D4Z4 DNA unit repeated 1-100 times where the repeat also contains the DUX4 open reading frame which is normally expressed in testis but is epigenetically repressed in somatic cells. At sizes greater than 10 repeats, the array adopts a repressed chromatin structure in somatic cells associated with high levels of CpG methylation and histone modifications. In FSHD1 patients, the D4Z4 array is shortened or contracted to 1-10 copies, at which point the region assumes a partially relaxed structure and DUX4 is transcriptionally de-repressed. The DUX4 gene lacks a polyA signal, but upon de-repression, the terminal DUX4 gene is stably expressed because the expressed RNA may be spliced to a polyA tail of the nearby pLAM locus. The DUX4 gene encodes a transcription factor that normally binds to a homeobox motif and regulates the expression of gene associated with stem cell and germline development. Mis-expression of DUX4 in skeletal muscle leads to cellular apoptosis and atophic myotube formation and can cause an upregulation of germline specific genes. Additionally, DUX4 expression leads to an inhibition of nonsense mediated RNA decay, meaning the cells accumulate a large number of RNA transcripts that normally would be degraded (Daxinger et al (2015) Curr Opin Genet Dev 33:56-61). Accordingly, the compositions and methods described herein can be used to repress (including inactivate) DUX4 expression for the treatment and/or prevention of FSHD and/or some or all of its symptoms.

In FSHD2 patients, clinical features are the same as for FSHD1 patients but the patients have more normal sized D4Z4 arrays. However, the D4Z4 arrays are hypomethylated in FSHD2 patients, suggesting an impairment in epigenetic regulation. In fact, it has been demonstrated that in 85% of FSHD2 patients, the disease is tied to a mutation in the Structural Maintenance of Chromosomes Hinge Domain Containing 1 (SMCHD1) gene. It appears that the SMCHD1 protein binds to telomeres, and may in fact bind to the D4Z4 array. The mutation thus may prevent or loosen the binding of the protein to the array and allow misexpression of DUX4 (Daxinger, ibid). Therefore, the artificial transcription factors and/or nucleases targeted to SMCHD1 are useful in treatment and/or prevention of FSHD2 and/or its symptoms. In some embodiments, the methods and compositions further comprise introduction of a wild-type SMCHD1 gene, wherein the wild-type SMCHD1 is either integrated into the genome using nuclease dependent targeted integration or the gene is maintained extrachromosomally.

Amyotrophic Lateral Sclerosis (ALS) is the most common adult-onset motor neuron disorder and is fatal for most patients less than three years from when the first symptoms appear. Generally, it appears that the development of ALS in approximately 90-95% of patients is completely random (sporadic ALS, sALS), with only 5-10% of patients displaying any kind of identified genetic risk (familial ALS, fALS). ALS has an annual incidence of 1-3 cases per 100,000 people. Mutations in several genes, including the C9orf72 (30-40% of patients), SOD1 (20-25%), TDP43/TARDBP, FUS1,(TDP43/TARDBP and Fus1 together are 5%), ANG, ALS2, SETX, and VAPB genes, cause familial ALS and contribute to the development of sporadic ALS. Mutations in the C9orf72gene are responsible for 30 to 40 percent of familial ALS in the United States and Europe and account for 5-10% of sporadic ALS. The C9orf72 mutations are typically hexanucleotide expansions of GGGGCC in the first intron of the C9orf72 gene and patients are typically heterozygous as this expansion results in an autosomal dominant phenotype. The pathology associated with this expansion (from approximately 30 copies in the wild type human genome to hundreds or even thousands in fALS patients) appears to be related to expression of both sense and anti-sense transcripts and to the formation of unusual structures in the DNA and to some type of RNA-mediated toxicity (Taylor (2014) Nature 507:175). Incomplete RNA transcripts of the expanded GGGGCC form nuclear foci in fALS patient cells and also the RNAs can also undergo repeat-associate non-ATP-dependent translation, resulting in the production of three proteins that are prone to aggregation (Gendron et al (2013) Acta Neuropathol 126:829). There is no ethnic or racial predisposition for ALS, and the incidence peaks in the population between 70 and 80 years of age, and the disease progresses rapidly (3-5 years) compared to other neurodegenerative disorders. Thus, the genetic modulators of C9orf72 as described herein can be used for the treatment and/or prevention of ALS in a subject in need thereof.

Frontotermporal dementia (FTD) is a progressive disorder of the brain that can affect behavior, language and movement. See, e.g., Benussi et al. (2015) Front Ag Neuro 7, art. 171. Mutations in C9orf72 have been implicated in FTD. Thus, the C9orf72-modulating compositions and methods described herein can be used to the treatment and/or prevention of FTD. In addition, FTD is also identified as a tauopathy, the methods and compositions described herein may further comprise administering one or more tau modulator (repressor) the FTD subject. See, e.g., U.S. Patent Publication No. 20180153921 for exemplary tau repressors. Zinc finger proteins linked to repression domains have been successfully used to preferentially repress the expression of expanded Htt alleles in cells derived from Huntington patients by binding to expanded tracts of CAG for the treatment of HD. See, also, U.S. Pat. Nos. 9,234,016 and 8,841,260. Similarly, the methods and compositions of the invention (TFs and/or nucleases targeted to ALS related genes such as C9orf72, SOD1, TDP43/TARDBP, FUS1) can be used to treat, delay or prevent ALS. For example, engineered DNA binding molecules (e.g. ZFPs, TALEs, guide RNAs) can be constructed to bind to the expansion tract of the C9orf72 disease associated allele and repress both sense and anti-sense expression. Alternatively, or in addition, a wild type version of C9orf72, lacking the abnormally expanded GGGGCC tract, may be inserted into the genome to allow for the normal expression of the gene product. These artificial transcription factors, nucleases, polynucleotides encoding these molecules and cells comprising these molecules or modified by these molecules, can be used to treat and/or prevent ALS.

Another genetic disease of the nervous system is Spinal Muscular Atrophy (SMA). SMA is the most frequent genetic cause of death in infants and toddlers (approximately 1 in 6-10,000 births) and involves progressive and symmetric muscle weakness involving the upper arm and leg muscles as well as the muscles of the head and trunk and intercostal muscles. Additionally, there is degeneration of the motor neurons in the spinal cord. SMA onset has been divided into three categories as follows: Type I, the most common with approximately 60% of SMA patients, has an onset at about 6 months of age and results in death by about 2 years; Type II has an onset between 6 and 18 months where the patient can have the ability to sit up, but not walk; and type III, which has an onset after 18 months, where the patients have some ability to walk for some amount of time. 95% of all types of SMA are tied to a homozygous loss of the survival motor neuron 1 (SMN1) protein. The SMN1 protein is required for the viability of all eukaryotic cells through its function as a co-factor in the assembly of the spliceosomal complex for RNA maturation (Talbot and Tizzano (2017) Gene Ther 24(9): 529-533). The severity of SMA can be offset by the expression of the SMN2 protein, which is nearly identical to SMN1 except for a single mutation that plays a role in the splicing of the RNA message. SMN2 is truncated however and rapidly degraded so while high expression of SMN2 may partially alleviate the loss of SMN1, it is not fully able to compensate (see Iascone et al (2015) F1000 Pri Rep 7:04). In fact, there appears to be an inverse correlation with the amount of SMN2 mRNA and the severity of the SMA disease. Since SMA is associated with a homozygous loss of the SMN1 gene, some researchers have tried introducing the SMN1 gene via an AAV9 viral vector in animal models of SMA (see Bevan et al (2011) Mol Ther 19(11):1971-1980). This early work showed that the gene could be delivered either through IV administration or through direct injection into the cerebral spinal fluid. However, penetration of the virus and complications relating to the crossing of the blood brain barrier still exist. Accordingly, the methods and compositions of the invention can be used to prevent or treat SMA. Engineered transcription factors specific for SNM2 may be designed to increase the expression of this gene. Engineered nucleases can also be used to cleave and correct the SMN2 mutation and cause stable expression by essentially turning it into the SMN1 gene. Furthermore, a wild type SMN1 cDNA may be inserted into the genome by targeted insertion using an engineered nuclease. The wild type SMN1 gene may be inserted into the endogenous SMN1 gene and thus be expressed under the regulation of the SMN1 promoter, or it may be inserted into a safe harbor gene (e.g. AAVS1). The gene may also be inserted via nuclease directed targeted integration into neuronal stem cells, where the engineered stem cells are then re-introduced into the patient such that the neurons that are derived from these stem cells function normally. Finally, the wild type SMN1 gene may be introduced into the brain via AAV delivery as a cDNA vector designed for episomal maintenance rather than integration into the genome. In this treatment modality, the cDNA vector would comprise a promoter for neural specific expression such as SYN1 or SMN1.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single-or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}. “Non-specific binding” refers to, non-covalent interactions that occur between any molecule of interest (e.g. an engineered nuclease) and a macromolecule (e.g. DNA) that are not dependent on target sequence.

A “DNA binding molecule” is a molecule that can bind to DNA. Such DNA binding molecule can be a polypeptide, a domain of a protein, a domain within a larger protein or a polynucleotide. In some embodiments, the polynucleotide is DNA, while in other embodiments, the polynucleotide is RNA. In some embodiments, the DNA binding molecule is a protein domain of a nuclease (e.g. the FokI domain), while in other embodiments, the DNA binding molecule is a guide RNA component of an RNA-guided nuclease (e.g. Cas9 or Cfp1).

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. The term “zinc finger nuclease” includes one ZFN as well as a pair of ZFNs that dimerize to cleave the target gene.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526. Zinc finger and TALE DNA-binding domains can be “engineered” to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496. The term “TALEN” includes one TALEN as well as a pair of TALENs that dimerize to cleave the target gene.

A “selected” zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

“TtAgo” is a prokaryotic Argonaute protein thought to be involved in gene silencing. TtAgo is derived from the bacteria Thermus thermophilus. See, e.g., Swarts et al (2014) Nature 507(7491):258-261, G. Sheng et al., (2013) Proc. Natl. Acad. Sci. U.S.A. 111, 652). A “TtAgo system” is all the components required including, for example, guide DNAs for cleavage by a TtAgo enzyme. “Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

Zinc finger binding domains or TALE DNA binding domains can be “engineered” to bind to a predetermined nucleotide sequence, for example via engineering (altering one or more amino acids) of the recognition helix region of a naturally occurring zinc finger protein or by engineering the RVDs of a TALE protein. Therefore, engineered zinc finger proteins or TALEs are proteins that are non-naturally occurring. Non-limiting examples of methods for engineering zinc finger proteins or TALEs are design and selection. A “designed” zinc finger protein or TALE is a protein not occurring in nature whose design/composition results principally from rational criteria. Rational criteria for design include application of substitution rules and computerized algorithms for processing information in a database storing information of existing ZFP designs and binding data. A “selected” zinc finger protein or TALE is a protein not found in nature whose production results primarily from an empirical process such as phage display, interaction trap or hybrid selection. See, for example, U.S. Pat. Nos. 8,586,526; 6,140,081; 6,453,242; 6,746,838; 7,241,573; 6,866,997; 7,241,574 and 6,534,261; see also WO 03/016496.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length. In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or noncoding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single-or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “multimerization domain”, (also referred to as a “dimerization domain” or “protein interaction domain”) is a domain incorporated at the amino, carboxy or amino and carboxy terminal regions of a ZFP TF or TALE TF. These domains allow for multimerization of multiple ZFP TF or TALE TF units such that larger tracts of trinucleotide repeat domains become preferentially bound by multimerized ZFP TFs or TALE TFs relative to shorter tracts with wild-type numbers of lengths. Examples of multimerization domains include leucine zippers. Multimerization domains may also be regulated by small molecules wherein the multimerization domain assumes a proper conformation to allow for interaction with another multimerization domain only in the presence of a small molecule or external ligand. In this way, exogenous ligands can be used to regulate the activity of these domains.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALE protein as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion molecules, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to upregulate gene expression. ZFPs fused to domains capable of regulating gene expression are collectively referred to as “ZFP-TFs” or “zinc finger transcription factors”, while TALEs fused to domains capable of regulating gene expression are collectively referred to as “TALE-TFs” or “TALE transcription factors.” When a fusion polypeptide in which a ZFP DNA-binding domain is fused to a cleavage domain (a “ZFN” or “zinc finger nuclease”), the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site. When a fusion polypeptide in which a TALE DNA-binding domain is fused to a cleavage domain (a “TALEN” or “TALE nuclease”), the TALE DNA-binding domain and the cleavage domain are in operative linkage if, in the fusion polypeptide, the TALE DNA-binding domain portion is able to bind its target site and/or its binding site, while the cleavage domain is able to cleave DNA in the vicinity of the target site. With respect to a fusion polypeptide in which a Cas DNA-binding domain is fused to an activation domain, the Cas DNA-binding domain and the activation domain are in operative linkage if, in the fusion polypeptide, the Cas DNA-binding domain portion is able to bind its target site and/or its binding site, while the activation domain is able to up-regulate gene expression. When a fusion polypeptide in which a Cas DNA-binding domain is fused to a cleavage domain, the Cas DNA-binding domain and the cleavage domain are in operative linkage if, in the fusion polypeptide, the Cas DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The terms “subject” and “patient” are used interchangeably and refer to mammals such as human patients and non-human primates, as well as experimental animals such as rabbits, dogs, cats, rats, mice, and other animals. Accordingly, the term “subject” or “patient” as used herein means any mammalian patient or subject to which the expression cassettes of the invention can be administered. Subjects of the present invention include those with a disorder or those at risk for developing a disorder.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Cancer and graft versus host disease are non-limiting examples of conditions that may be treated using the compositions and methods described herein. Thus, “treating” and “treatment includes:
(i) preventing the disease or condition from occurring in a mammal, in particular, when such mammal is predisposed to the condition but has not yet been diagnosed as having it;
(ii) inhibiting the disease or condition, i.e., arresting its development;
(iii) relieving the disease or condition, i.e., causing regression of the disease or condition; and/or
(iv) relieving or eliminating the symptoms resulting from the disease or condition, i.e., relieving pain with or without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment in the mammal, preferably a human. The amount of a composition of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

### DNA-Binding Domains

The methods described herein make use of compositions, for example gene-modulating transcription factors, comprising a DNA-binding domain that specifically binds to a target sequence (e.g., a target site of 9-20 or more contiguous or non-contiguous nucleotides) in an endogenous DUX4, C9orf72, SMN1, SMN2, UBE34, or Ube34-ATS gene. Any polynucleotide or polypeptide DNA-binding domain can be used in the compositions and methods disclosed herein, for example DNA-binding proteins (e.g., ZFPs or TALEs) or DNA-binding polynucleotides (e.g., single guide RNAs). Thus, genetic repressors of DUX4, C9orf72, SMN1, SMN2, UBE34, or Ube34-ATS genes are described.

In certain embodiments, the repressor, or DNA binding domain therein, comprises a zinc finger protein. Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

DUX4, C9orf72, SMN1, SMN2, UBE34, and Ube34-ATS-targeted ZFPs typically include at least one zinc finger but can include a plurality of zinc fingers (e.g., 2, 3, 4, 5, 6 or more fingers). In certain embodiments, the ZFPs include at least three fingers. Certain of the ZFPs include four, five or six fingers, while some ZFPs include 8, 9, 10, 11 or 12 fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more regulatory domains, which domains can be transcriptional activation or repression domains. In some embodiments, the fusion protein comprises two ZFP DNA binding domains linked together. These zinc finger proteins can thus comprise 8, 9, 10, 11, 12 or more fingers. In some embodiments, the two DNA binding domains are linked via an extendable flexible linker such that one DNA binding domain comprises 4, 5, or 6 zinc fingers and the second DNA binding domain comprises an additional 4, 5, or 5 zinc fingers. In some embodiments, the linker is a standard inter-finger linker such that the finger array comprises one DNA binding domain comprising 8, 9, 10, 11 or 12 or more fingers. In other embodiments, the linker is an atypical linker such as a flexible linker. The DNA binding domains are fused to at least one regulatory domain and can be thought of as a ‘ZFP-ZFP-TF’ architecture. Specific examples of these embodiments can be referred to as “ZFP-ZFP-KOX” which comprises two DNA binding domains linked with a flexible linker and fused to a KOX repressor and “ZFP-KOX-ZFP-KOX” where two ZFP-KOX fusion proteins are fused together via a linker.

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128.

“Two handed” zinc finger proteins are those proteins in which two clusters of zinc finger DNA binding domains are separated by intervening amino acids so that the two zinc finger domains bind to two discontinuous target sites. An example of a two handed type of zinc finger binding protein is SIP1, where a cluster of four zinc fingers is located at the amino terminus of the protein and a cluster of three fingers is located at the carboxyl terminus (see Remacle et al, (1999) EMBO Journal 18 (18): 5073-5084). Each cluster of zinc fingers in these proteins is able to bind to a unique target sequence and the spacing between the two target sequences can comprise many nucleotides. Two-handed ZFPs may include a functional domain, for example fused to one or both of the ZFPs. Thus, it will be apparent that the functional domain may be attached to the exterior of one or both ZFPs or may be positioned between the ZFPs (attached to both ZFPs). In certain embodiments, the ZFP comprises a ZFP as shown in Table 1.

In certain embodiments, the DNA-binding domain comprises a naturally occurring or engineered (non-naturally occurring) TAL effector (TALE) DNA binding domain. See, e.g., U.S. Pat. No. 8,586,526, incorporated by reference in its entirety herein. In certain embodiments, the TALE DNA-binding protein comprises binds to 12, 13, 14, 15, 16, 17, 18, 19, 20 or more contiguous nucleotides of a target site as shown in Table 1. The RVDs of the TALE DNA-binding protein that binds to a target site may be naturally occurring or non-naturally occurring RVDs. See, U.S. Pat. Nos. 8,586,5226 and 9,458,205.

The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218:127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove (2009) Science 326:1501 and Boch et al (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and NG binds to T. These DNA binding repeats have been assembled into proteins with new combinations and numbers of repeats, to make artificial transcription factors that are able to interact with new sequences. In addition, U.S. Pat. No. 8,586,526 and U.S. Publication No. 20130196373, incorporated by reference in their entireties herein, describe TALEs with N-cap polypeptides, C-cap polypeptides (e.g., +63, +231 or +278) and/or novel (atypical) RVDs. Such TALEs are described in U.S. Pat. No. 8,586,526 and 9,458,205, incorporated by reference in their entireties.

In certain embodiments, the DNA binding domains include a dimerization and/or multimerization domain, for example a coiled-coil (CC) and dimerizing zinc finger (DZ). See, U.S. Patent Publication No. 20130253040.

In still further embodiments, the DNA-binding domain comprises a single-guide RNA of a CRISPR/Cas system, for example sgRNAs as disclosed in U.S. Patent Publication No. 20150056705.

Compelling evidence has recently emerged for the existence of an RNA-mediated genome defense pathway in archaea and many bacteria that has been hypothesized to parallel the eukaryotic RNAi pathway (for reviews, see Godde and Bickerton, 2006. J. Mol. Evol. 62:718-729; Lillestol et al., 2006. Archaea 2:59-72; Makarova et al., 2006. Biol. Direct 1:7.; Sorek et al., 2008. Nat. Rev. Microbiol. 6:181-186). Known as the CRISPR-Cas system or prokaryotic RNAi (pRNAi), the pathway is proposed to arise from two evolutionarily and often physically linked gene loci: the CRISPR (clustered regularly interspaced short palindromic repeats) locus, which encodes RNA components of the system, and the cas (CRISPR-associated) locus, which encodes proteins (Jansen et al., 2002. Mol. Microbiol. 43:1565-1575; Makarova et al., 2002. Nucleic Acids Res. 30:482-496; Makarova et al., 2006. Biol. Direct 1:7; Haft et al., 2005. PLOS Comput. Biol. 1:e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (Makarova et al., 2006. Biol. Direct 1:7). The CRISPR-associated (cas) genes are often associated with CRISPR repeat-spacer arrays. More than forty different Cas protein families have been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

The Type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system.

Type II CRISPR systems have been found in many different bacteria. BLAST searches on publically available genomes by Fonfara et al ((2013) Nuc Acid Res 42(4):2377-2590) found Cas9 orthologs in 347 species of bacteria. Additionally, this group demonstrated in vitro CRISPR/Cas cleavage of a DNA target using Cas9orthologs from S. pyogenes, S. mutans, S. therophilus, C. jejuni, N. meningitides, P. multocida and F. novicida. Thus, the term “Cas9” refers to an RNA guided DNA nuclease comprising a DNA binding domain and two nuclease domains, where the gene encoding the Cas9 may be derived from any suitable bacteria.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The Cas 9 nuclease can be engineered such that only one of the nuclease domains is functional, creating a Cas nickase (see Jinek et al, ibid). Nickases can be generated by specific mutation of amino acids in the catalytic domain of the enzyme, or by truncation of part or all of the domain such that it is no longer functional. Since Cas 9 comprises two nuclease domains, this approach may be taken on either domain. A double strand break can be achieved in the target DNA by the use of two such Cas 9 nickases. The nickases will each cleave one strand of the DNA and the use of two will create a double strand break.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam, ibid) and has been useful for zebrafish embryo genomic editing in vivo (see Hwang et al (2013) Nature Biotechnology 31 (3):227) with editing efficiencies similar to ZFNs and TALENs.

The primary products of the CRISPR loci appear to be short RNAs that contain the invader targeting sequences, and are termed guide RNAs or prokaryotic silencing RNAs (psiRNAs) based on their hypothesized role in the pathway (Makarova et al., 2006. Biol. Direct 1:7; Hale et al., 2008. RNA, 14: 2572-2579). RNA analysis indicates that CRISPR locus transcripts are cleaved within the repeat sequences to release ^{˜}60- to 70-nt RNA intermediates that contain individual invader targeting sequences and flanking repeat fragments (Tang et al. 2002. Proc. Natl. Acad. Sci. 99: 7536-7541; Tang et al., 2005. Mol. Microbiol. 55: 469-481; Lillestol et al. 2006. Archaea 2: 59-72; Brouns et al. 2008. Science 321: 960-964; Hale et al, 2008. RNA, 14: 2572-2579). In the archaeon Pyrococcus furiosus, these intermediate RNAs are further processed to abundant, stable ^{˜}35- to 45-nt mature psiRNAs (Hale et al. 2008. RNA, 14: 2572-2579).

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337:816 and Cong et al (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam ibid) and has been useful for zebrafish embryo genomic editing in vivo (see Hwang et al (2013) Nature Biotechnology 31 (3):227) with editing efficiencies similar to ZFNs and TALENs.

Chimeric or sgRNAs can be engineered to comprise a sequence complementary to any desired target. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In certain embodiments, the sgRNA comprises a sequence that binds to 12, 13, 14, 15, 16, 17, 18, 19, 20 or more contiguous nucleotides of a target site within a disease-associated gene (e.g., DUX4, C9orf72, SMN1, SMN2, UBE34, or Ube34-ATS). In some embodiments, the RNAs comprise 22 bases of complementarity to a target and of the form G[n19], followed by a protospacer-adjacent motif (PAM) of the form NGG or NAG for use with a S. pyogenes CRISPR/Cas system. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20] GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence the conforms to the G[n20] GG formula. Along with the complementarity region, an sgRNA may comprise additional nucleotides to extend to tail region of the tracrRNA portion of the sgRNA (see Hsu et al (2013) Nature Biotech doi:10.1038/nbt.2647). Tails may be of +67 to +85 nucleotides, or any number therebetween with a preferred length of +85 nucleotides. Truncated sgRNAs may also be used, “tru-gRNAs” (see Fu et al, (2014) Nature Biotech 32(3): 279). In tru-gRNAs, the complementarity region is diminished to 17 or 18 nucleotides in length.

Further, alternative PAM sequences may also be utilized, where a PAM sequence can be NAG as an alternative to NGG (Hsu 2014, ibid) using a S. pyogenes Cas9. Additional PAM sequences may also include those lacking the initial G (Sander and Joung (2014) Nature Biotech 32(4):347). In addition to the S. pyogenes encoded Cas9 PAM sequences, other PAM sequences can be used that are specific for Cas9 proteins from other bacterial sources. For example, the PAM sequences shown below (adapted from Sander and Joung, ibid, and Esvelt et al, (2013) Nat Meth 10(11):1116) are specific for these Cas9 proteins:

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |

Thus, a suitable target sequence for use with a S. pyogenes CRISPR/Cas system can be chosen according to the following guideline: [n17, n18, n19, or n20] (G/A) G. Alternatively the PAM sequence can follow the guideline G [n17, n18, n19, n20] (G/A) G. For Cas9 proteins derived from non-S. pyogenes bacteria, the same guidelines may be used where the alternate PAMs are substituted in for the S. pyogenes PAM sequences.

Most preferred is to choose a target sequence with the highest likelihood of specificity that avoids potential off target sequences. These undesired off target sequences can be identified by considering the following attributes: i) similarity in the target sequence that is followed by a PAM sequence known to function with the Cas9 protein being utilized; ii) a similar target sequence with fewer than three mismatches from the desired target sequence; iii) a similar target sequence as in ii), where the mismatches are all located in the PAM distal region rather than the PAM proximal region (there is some evidence that nucleotides 1-5 immediately adjacent or proximal to the PAM, sometimes referred to as the ‘seed’ region (Wu et al (2014) Nature Biotech doi:10.1038/nbt2889) are the most critical for recognition, so putative off target sites with mismatches located in the seed region may be the least likely be recognized by the sg RNA); and iv) a similar target sequence where the mismatches are not consecutively spaced or are spaced greater than four nucleotides apart (Hsu 2014, ibid). Thus, by performing an analysis of the number of potential off target sites in a genome for whichever CRIPSR/Cas system is being employed, using these criteria above, a suitable target sequence for the sgRNA may be identified.

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al. (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired IIDR recombination event has taken place. Thus, in the methods and compositions described herein, it is understood that the term “‘Cas” includes both Cas9 and Cfp1 proteins. Thus, as used herein, a “CRISPR/Cas system” refers both CRISPR/Cas and/or CRISPR/Cfp1 systems, including both nuclease, nickase and/or transcription factor systems.

In some embodiments, other Cas proteins may be used. Some exemplary Cas proteins include Cas9, Cpf1 (also known as Cas12a), C2c1, C2c2 (also known as Cas13a), C2c3, Cas1, Cas2, Cas4, CasX and CasY; and include engineered and natural variants thereof (Burstein et al. (2017) Nature 542:237-241) for example HF1/spCas9 (Kleinstiver et al. (2016) Nature 529:490-495; Cebrian-Serrano and Davies (2017) Mamm Genome 28(7):247-261); split Cas9 systems (Zetsche et al. (2015) Nat Biotechnol 33(2):139-142), trans-spliced Cas9 based on an intein-extein system (Troung et al. (2015) Nucl Acid Res 43(13):6450-8); mini-SaCas9 (Ma et al. (2018) ACS Synth Biol 7(4):978-985). Thus, in the methods and compositions described herein, it is understood that the term “‘Cas” includes all Cas variant proteins, both natural and engineered. Thus, as used herein, a “CRISPR/Cas system” refers to any CRISPR/Cas system, including both nuclease, nickase and/or transcription factor systems.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to specific genes (including safe harbor genes) are disclosed for example, in U.S. Publication No. 20150056705.

Thus, the genetic modulators described herein (artificial transcription factors, nucleases, etc.) comprises a DNA-binding molecule in that specifically binds to a target site in any gene, and any DNA-binding molecule can be used.

### Gene Modulators

The DNA-binding domains may be fused to or otherwise associate with any additional molecules (e.g., polypeptides) for use in the methods described herein. In certain embodiments, the methods employ fusion molecules comprising at least one DNA-binding molecule (e.g., ZFP, TALE or single guide RNA) and a heterologous regulatory (functional) domain (or functional fragment thereof), for instance artificial transcription factors (activators or repressors) comprising a DNA-binding domain that binds to a target site in the rare disease-associate gene and a transcriptional regulatory domain.

In certain embodiments, the functional domain of the gene modulator comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, kinases, phosphatases, polymerases, endonucleases) and their associated factors and modifiers. See, e.g., U.S. Publication No. 20130253040, incorporated by reference in its entirety herein.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, October 1, Oct-2A, Sp1, AP-2, and CTF1 (Seipel et al., EMBO J. 11, 4961-4968 (1992) as well as p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5,-6,-7, and -8, CPRF1, CPRF4, MYC-RP/GP, and TRAB1. See, for example, Ogawa et al. (2000) Gene 245:21-29; Okanami et al. (1996) Genes Cells 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15,348-15,353.

Exemplary repression domains that can be used to make gene repressors include, but are not limited to, KRAB A/B, KOX, TGF-beta-inducible early gene (TIEG), v-crbA, SID, MBD2, MBD3, members of the DNMT family (e.g., DNMT1, DNMT3A, DNMT3B), Rb, and MeCP2. See, for example, Bird et al. (1999) Cell 99:451-454; Tyler et al. (1999) Cell 99:443-446; Knoepfler et al. (1999) Cell 99:447-450; and Robertson et al. (2000) Nature Genet. 25:338-342. Additional exemplary repression domains include, but are not limited to, ROM2 and AtHD2A. See, for example, Chem et al. (1996) Plant Cell 8:305-321; and Wu et al. (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Berndsen and Denu (2008) Curr Opin Struct Biol 18(6):682-689). In other instances the domain is a histone deacetylase (HDAC) such as the class I (HDAC-1, 2, 3, and 8), class II (HDAC IIA (HDAC-4, 5, 7 and 9), HDAC IIB (HDAC 6 and 10)), class IV (HDAC-11), class III (also known as sirtuins (SIRTs); SIRT1-7) (see Mottamal et al (2015) Molecules 20(3):3898-3941). Another domain that is used in some embodiments is a histone phosphorylase or kinase, where examples include MSK1, MSK2, ATR, ATM, DNA-PK, Bub1, VprBP, IKK-α, PKCβ1, Dik/Zip, JAK2, PKC5, WSTF and CK2. In some embodiments, a methylation domain is used and may be chosen from a groups such as Ezh2, PRMT1/6, PRMT5/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h. Domains involved in sumoylation and biotinylation (Lys9, 13, 4, 18 and 12) may also be used in some embodiments (review see Kousarides (2007) Cell 128:693-705).

Heterologous regulatory (functional) domain (or functional fragment thereof) associated with the DNA-binding domains described herein (e.g., ZFPs, TALES, sgRNAs, etc.) therefore include, but are not limited to, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases, topoisomerases, helicases, ligases, deubiquitinases, kinases, phosphatases, polymerases, endonucleases) and their associated factors and modifiers. Such fusion molecules include transcription factors comprising the DNA-binding domains described herein and a transcriptional regulatory domain as well as nucleases comprising the DNA-binding domains and one or more nuclease domains.

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al. (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

The fusion molecule may be formulated with a pharmaceutically acceptable carrier, as is known to those of skill in the art. See, for example, Remington's Pharmaceutical Sciences, 17th ed., 1985; and co-owned WO 00/42219.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

In certain embodiments, the fusion molecule comprises a DNA-binding domain and a nuclease domain to create functional entities that are able to recognize their intended nucleic acid target through their engineered (ZFP or TALE) DNA binding domains and create nucleases (e.g., zinc finger nuclease or TALE nucleases) cause the DNA to be cut near the DNA binding site via the nuclease activity. This cleavage results in inactivation (repression) of a targeted gene. Thus, gene repressors also include targeted nucleases.

It will be clear to those of skill in the art that, in the formation of a fusion protein (or a nucleic acid encoding same) between a DNA-binding domain and a functional domain, either an activation domain or a molecule that interacts with an activation domain is suitable as a functional domain. Essentially any molecule capable of recruiting an activating complex and/or activating activity (such as, for example, histone acetylation) to the target gene is useful as an activating domain of a fusion protein. Insulator domains, localization domains, and chromatin remodeling proteins such as ISWI-containing domains and/or methyl binding domain proteins suitable for use as functional domains in fusion molecules are described, for example, in U.S. Pat. No. 7,053,264.

Thus, the methods and compositions described herein are broadly applicable and may involve any artificial nuclease or transcription factor of interest. Non-limiting examples of nucleases include meganucleases, TALENs and zinc finger nucleases. The nuclease may comprise heterologous DNA-binding and cleavage domains (e.g., zinc finger nucleases; TALENs; meganuclease DNA-binding domains with heterologous cleavage domains) or, alternatively, the DNA-binding domain of a naturally-occurring nuclease may be altered to bind to a selected target site (e.g., a meganuclease that has been engineered to bind to site different than the cognate binding site). Non-limiting examples of artificial transcription factors include ZFP-TFs, TALE-TFs and/or CRISPR/Cas-TFs.

The nuclease domain may be derived from any nuclease, for example any endonuclease or exonuclease. Non-limiting examples of suitable nuclease (cleavage) domains that may be fused to target DNA-binding domains as described herein include domains from any restriction enzyme, for example a Type IIS Restriction Enzyme (e.g., FokI). In certain embodiments, the cleavage domains are cleavage half-domains that require dimerization for cleavage activity. See, e.g., U.S. Pat. Nos. 8,586,526; 8,409,861 and 7,888,121, incorporated by reference in their entireties herein. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing.

The nuclease domain may also be derived any meganuclease (homing endonuclease) domain with cleavage activity may also be used with the nucleases described herein, including but not limited to I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. In certain embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the meganuclease (e.g., TevI) nuclease domain (see Beurdeley et al (2013) Nat Comm: 1-8 DOI: 10.1038/ncomms2782). Any TALENs may be used in combination with additional TALENs (e.g., one or more TALENs (cTALENs or FokI-TALENs) with one or more mega-TALs) or other DNA cleavage enzymes. In certain embodiments, the nuclease comprises a meganuclease (homing endonuclease) or a portion thereof that exhibits cleavage activity. Naturally-occurring meganucleases recognize 15-40 base-pair cleavage sites and are commonly grouped into four families: the LAGLIDADG family (SEQ ID NO: 34), the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res: 1-13, doi: 10.1093/nar/gkt1224).

In addition, the nuclease domain of the meganuclease may also exhibit DNA-binding functionality. Any TALENs may be used in combination with additional TALENs (e.g., one or more TALENs (cTALENs or FokI-TALENs) with one or more mega-TALs) and/or ZFNs.

In addition, cleavage domains may include one or more alterations as compared to wild-type, for example for the formation of obligate heterodimers that reduce or eliminate off-target cleavage effects. See, e.g., U.S. Pat. Nos. 7,914,796; 8,034,598; and 8,623,618, incorporated by reference in their entireties herein.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95:10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a zinc finger binding domain and two Fok I cleavage half-domains can also be used. Parameters for targeted cleavage and targeted sequence alteration using zinc finger-Fok I fusions are provided elsewhere in this disclosure.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Pat. Nos. 7,914,796; 8,034,598 and 8,623,618; and U.S. Patent Publication No. 20110201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Pat. Nos. 7,914,796 and 8,034,598, the disclosures of which are incorporated by reference in their entireties for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). See, e.g., U.S. Pat. Nos. 7,914,796; 8,034,598 and 8,623,618, the disclosures of which are incorporated by reference in its entirety for all purposes. In other embodiments, the engineered cleavage half domain comprises the “Sharkey” and/or “Sharkey” mutations (see Guo et al, (2010) J. Mol. Biol. 400(1):96-107).

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in as described in U.S. Pat. No. 8,563,314.

In certain embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLOS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation’, (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system and serve roles in functions such as insertion of the alien DNA etc.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems are disclosed for example, in U.S. Publication No. 20150056705.

The nuclease(s) may make one or more double-stranded and/or single-stranded cuts in the target site. In certain embodiments, the nuclease comprises a catalytically inactive cleavage domain (e.g., FokI and/or Cas protein). See, e.g., U.S. Pat. No. 9,200,266; 8,703,489 and Guillinger et al. (2014) Nature Biotech. 32(6):577-582. The catalytically inactive cleavage domain may, in combination with a catalytically active domain act as a nickase to make a single-stranded cut. Therefore, two nickases can be used in combination to make a double-stranded cut in a specific region. Additional nickases are also known in the art, for example, McCaffrey et al. (2016) Nucleic Acids Res. 44(2):e11. doi: 10.1093/nar/gkv878. Epub 2015 Oct. 19.

Nucleases as described herein may generate double-or single-stranded breaks in a double-stranded target (e.g., gene). The generation of single-stranded breaks (“nicks”) is described, for example in U.S. Pat. Nos. 8,703,489 and 9,200,266, incorporated herein by reference which describes how mutation of the catalytic domain of one of the nucleases domains results in a nickase.

Thus, a nuclease (cleavage) domain or cleavage half-domain can be any portion of a protein that retains cleavage activity, or that retains the ability to multimerize (e.g., dimerize) to form a functional cleavage domain.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Publication No. 20090111119. Nuclease expression constructs can be readily designed using methods known in the art.

Expression of the fusion proteins (or component thereof) may be under the control of a constitutive promoter or an inducible promoter, for example the galactokinase promoter which is activated (de-repressed) in the presence of raffinose and/or galactose and repressed in presence of glucose. Non-limiting examples of preferred promoters include the neural specific promoters NSE, Synapsin, CAMKiia and MECPs. Non-limiting examples of ubiquitious promoters include CAS and Ubc. Further embodiments include the use of self-regulating promoters (via the inclusion of high affinity binding sites for the target DNA-binding domain) as described in US Publication No. 20150267205).

### Delivery

The transcription factors, nucleases and/or polynucleotides (e.g., gene modulators) and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, Ad vector, etc.). In preferred embodiments, the genetic modulator (e.g., repressor) is delivered using an AAV vector, including but not limited to an AAV9 vector (or pseuotyped vector thereof) (see U.S. Pat. No. 7,198,951) or an AAV vector as described in U.S. Pat. No. 9,585,971.

Methods of delivering proteins comprising zinc finger proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 8,586,526; 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences. Thus, when one or more modulators (e.g., repressors) are introduced into the cell, the sequences encoding the protein components and/or polynucleotide components may be carried on the same vector or on different vectors. When multiple vectors are used, each vector may comprise a sequence encoding one or multiple gene modulators (e.g,. repressors) or components thereof.

Conventional viral and non-viral based gene transfer methods can be used to introduce nucleic acids encoding engineered gene modulators in cells (e.g., mammalian cells) and target tissues. Such methods can also be used to administer nucleic acids encoding such repressors (or components thereof) to cells in vitro. In certain embodiments, nucleic acids encoding the repressors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, naked RNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See US patents U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Maryland), BTX Molecular Delivery Systems (Holliston, MA) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™ and Lipofectamine™ RNAiMAX). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology 27(7):643).

The use of RNA or DNA viral based systems for the delivery of nucleic acids encoding engineered ZFPs, TALEs or CRISPR/Cas systems take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs, TALEs or CRISPR/Cas systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon mouse leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6, AAV8AAV 8.2, AAV9, and AAV rh10 and pseudotyped AAV such as AAV2/8, AAV2/5 and AAV2/6 can also be used in accordance with the present invention. AAV serotypes capable of crossing the blood-brain barrier can also be used in accordance with the present invention (see e.g. U.S. Pat. No. 9,585,971). In preferred embodiments, AAV9 vector (including variants and pseudotypes of AAV9) is used.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

Purification of AAV particles from a 293 or baculovirus system typically involves growth of the cells which produce the virus, followed by collection of the viral particles from the cell supernatant or lysing the cells and collecting the virus from the crude lysate. AAV is then purified by methods known in the art including ion exchange chromatography (e.g. see U.S. Pat. No. 7,419,817 and 6,989,264), ion exchange chromatography and CsCl density centrifugation (e.g. PCT publication WO2011094198A10), immunoaffinity chromatography (e.g. WO2016128408) or purification using AVB Sepharose (e.g. GE Healthcare Life Sciences).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney mouse leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion, including direct injection into the brain) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In certain embodiments, the compositions as described herein (e.g., polynucleotides and/or proteins) are delivered directly in vivo. The compositions (cells, polynucleotides and/or proteins) may be administered directly into the central nervous system (CNS), including but not limited to direct injection into the brain or spinal cord. One or more areas of the brain may be targeted, including but not limited to, the hippocampus, the substantia nigra, the nucleus basalis of Meynert (NBM), the striatum and/or the cortex. Alternatively or in addition to CNS delivery, the compositions may be administered systemically (e.g., intravenous, intraperitoneal, intracardial, intramuscular, intrathecal, subdermal, and/or intracranial infusion). Methods and compositions for delivery of compositions as described herein directly to a subject (including directly into the CNS) include but are not limited to direct injection (e.g., stereotactic injection) via needle assemblies. Such methods are described, for example, in U.S. Pat. Nos. 7,837,668; 8,092,429, relating to delivery of compositions (including expression vectors) to the brain and U.S. Patent Publication No. 20060239966, incorporated herein by reference in their entireties.

The effective amount to be administered will vary from patient to patient and according to the mode of administration and site of administration. Accordingly, effective amounts are best determined by the physician administering the compositions and appropriate dosages can be determined readily by one of ordinary skill in the art. After allowing sufficient time for integration and expression (typically 4-15 days, for example), analysis of the serum or other tissue levels of the therapeutic polypeptide and comparison to the initial level prior to administration will determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to annually to every several years.

To deliver the compositions described herein using adeno-associated viral (AAV) vectors directly to the human brain, a dose range of 1×10¹⁰-5×10¹⁵ (or any value therebetween) vector genome per striatum can be applied. As noted, dosages may be varied for other brain structures and for different delivery protocols. Methods of delivering AAV vectors directly to the brain are known in the art. See, e.g., U.S. Pat. Nos. 9,089,667; 9,050,299; 8,337,458; 8,309,355; 7,182,944; 6,953,575; and 6,309,634.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with at least one gene modulator (e.g., repressor) or component thereof and re-infused back into the subject organism (e.g., patient). In a preferred embodiment, one or more nucleic acids of the gene modulator (e.g., repressor) are delivered using AAV9. In other embodiments, one or more nucleic acids of the gene modulator (e.g., repressor) are delivered as mRNA. Also preferred is the use of capped mRNAs to increase translational efficiency and/or mRNA stability. Especially preferred are ARCA (anti-reverse cap analog) caps or variants thereof. See U.S. Pat. No. 7,074,596 and 8,153,773, incorporated by reference herein in their entireties. Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX-or BAK-specific TALENs or ZFNs (see, U.S. Pat. No. 8,597,912) in the stem cells, or those that are disrupted in a caspase, again using caspase-6 specific ZFNs for example. These cells can be transfected with the ZFP TFs or TALE TFs that are known to regulate a target gene.

Vectors (e.g., retroviruses, adenoviruses, liposomes, etc.) containing therapeutic ZFP nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34⁺ cells, include adenovirus Type 35.

Vectors suitable for introduction of transgenes into immune cells (e.g., T-cells) include non-integrating lentivirus vectors. See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NS0, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used. In a preferred embodiment, the methods and composition are delivered directly to a brain cell, for example in the striatum.

### Models of CNS disorders

Studies of CNS disorders can be carried out in animal model systems such as non-human primates (e.g., Parkinson's Disease (Johnston and Fox (2015) Curr Top Behav Neurosci 22:221-35); Amyotrophic lateral sclerosis (Jackson et al, (2015) J. Med Primatol: 44(2):66-75), Huntington's Disease (Yang et al (2008) Nature 453(7197):921-4); Alzheimer's Disease (Park et al (2015) Int J Mol Sci 16(2):2386-402); Seizure (Hsiao et al (2016) EBioMed 9:257-77), canines (e.g. MPS VII (Gurda et al (2016) Mol Ther 24(2):206-216); Alzheimer's Disease (Schutt et al (J Alzheimers Dis 52(2):433-49); Seizure (Varatharajah et al (2017) Int J Neural Syst 27(1):1650046) and mice (e.g. Seizure (Kadiyala et al (2015) Epilepsy Res 109:183-96); Alzheimer's Disease (Li et al (2015) J Alzheimers Dis Parkin 5(3) doi 10:4172/2161-0460), (review: Webster et al (2014) Front Genet 5 art 88, doi:10.3389f/gene.2014.00088). These models may be used even when there is no animal model that completely recapitulates a CNS disease as they may be useful for investigating specific symptom sets of a disease. The models may be helpful in determining efficacy and safety profiles of a therapeutic methods and compositions (genetic repressors) described herein.

### Applications

Gene modulators as described herein comprising DUX4, C9orf72, UBE34, Ube3a-ATS, SMN1, or SMN2 binding molecules (e.g., ZFPs, TALEs, CRISPR/Cas systems, Ttago, etc.) as described herein, and the nucleic acids encoding them, can be used for a variety of applications. These applications include therapeutic methods in which a DUX4, C9orf72, UBE34, Ube3a-ATS, SMN1, or SMN2-binding molecule (including a nucleic acid encoding a DNA-binding protein) is administered to a subject using a viral (e.g., AAV) or non-viral vector and used to modulate the expression of a target gene within the subject. The modulation can be in the form of repression, for example, repression of C9orf72 (e.g., mutant) expression that is contributing to an ALS or FTD disease state or repression or Ube3a-ATS expression that is contributing to an AS disease state. Alternatively, the modulation can be in the form of activation when activation of expression or increased expression of an endogenous cellular gene can ameliorate a diseased state. In still further embodiments, the modulation can be repression via cleavage (e.g., by one or more nucleases), for example, for inactivation of a DUX4, C9orf72, UBE34, Ube3a-ATS, SMN1, or SMN2 gene. As noted above, for such applications, the target-binding molecules, or more typically, nucleic acids encoding them are formulated with a pharmaceutically acceptable carrier as a pharmaceutical composition.

The DUX4, C9orf72, UBE34, Ube3a-ATS, SMN1, or SMN2-binding molecules, or vectors encoding them, alone or in combination with other suitable components (e.g. liposomes, nanoparticles or other components known in the art), can be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically, intracranially or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient should be sufficient to effect a beneficial therapeutic response in the patient over time. The dose is determined by the efficacy and K_{d} of the particular gene targeting molecule employed, the target cell, and the condition of the patient, as well as the body weight or surface area of the patient to be treated. The size of the dose also is determined by the existence, nature, and extent of any adverse side-effects that accompany the administration of a particular compound or vector in a particular patient.

The following Examples relate to exemplary embodiments of the present disclosure. It will be appreciated that this is for purposes of exemplification only and that other gene-modulators (e.g., repressors) can be used, including, but not limited to, TALE-TFs, a CRISPR/Cas system, additional ZFPs, ZFNs, TALENs, additional CRISPR/Cas systems, homing endonucleases (meganucleases) with engineered DNA-binding domains. It will be apparent that these modulators can be readily obtained using methods known to the skilled artisan to bind to the target sites as exemplified below.

### EXAMPLES

### Example 1: Artificial Transcription Factors

Zinc finger proteins, TALEs and sgRNAs targeted to DUX4, C9orf72, UBE34, Ube3a-ATS, SMN1, or SMN2 are engineered essentially as described in U.S. Pat. Nos. 6,534,261 & 8,586,526 and U.S. Patent Publication Nos. 20150056705; 20110082093; 20130253040; & 20150335708. A set of repressors are also made to target DUX4, C9orf72, UBE34, Ube3a-ATS, SMN1, or SMN2 sequences in both mice and humans. The repressors are evaluated by standard SELEX analysis and are shown to bind to their target sites. A linker was used to link the ZFP DNA binding domain to the transcriptional repressor, where the linker had the following amino acid sequence: LRQKDAARGS (SEQ ID NO: 33). Exemplary ZFPs targeted to C9orf72 are shown below in Table 1. Their target sites are shown in capital letters in SEQ ID NO:1 and SEQ ID NO:2. The capitalized sequences, nine in total, are SEQ ID NOs: 37-45, respectively, in order of appearance. All ZFPs in Table 1 were shown to bind to their target sites.

TABLE 1
C9orf72 ZFP designs

| SBS#/ | | | | | | |
|---|---|---|---|---|---|---|
| target site | F1 | F2 | F3 | F4 | F5 | F6 |
| SBS# 74949 | DRSDLSR | RSTHLVR | DRSDLSR | RSTHLVR | DRSDLSR | RSTHLVR |
| taGGGGCCGGGGCCGG | (SEQ ID | (SEQ ID | (SEQ ID | (SEQ ID | (SEQ ID | (SEQ ID |
| GGCCggggcgtg | NO: 3) | NO: 4) | NO: 3) | NO: 4) | NO: 3) | NO: 4) |
| (SEQ ID NO: 1) | | | | | | |
| SBS# 74951 | DRSDLSR | RSAHLSR | DRSDLSR | RSAHLSR | DRSDLSR | RSAHLSR |
| taGGGGCCGGGGCCGG | (SEQ ID | (SEQ ID | (SEQ ID | (SEQ ID | (SEQ ID | (SEQ ID |
| GGCCggggcgtg | NO: 3) | NO: 5) | NO: 3) | NO: 5) | NO: 3) | NO: 5) |
| (SEQ ID NO: 1) | | | | | | |
| SBS#74954 | ERGDLKR | RSAHLSR | ERGDLKR | RSAHLSR | ERGDLKR | RSAHLSR |
| taGGGGCCGGGGCCGG | (SEQ ID | (SEQ ID | (SEQ ID | (SEQ ID | (SEQ ID | (SEQ ID |
| GGCCggggcgtg | NO: 6) | NO: 5) | NO: 6) | NO: 5) | NO: 6) | NO: 5) |
| (SEQ ID NO: 1) | | | | | | |
| SBS#74955 | ERGTLAR | RSAHLSR | ERGTLAR | RSAHLSR | ERGTLAR | RSAHLSR |
| taGGGGCCGGGGCCGG | (SEQ ID | (SEQ ID | (SEQ ID | (SEQ ID | (SEQ ID | (SEQ ID |
| GGCCggggcgtg | NO: 7) | NO: 5) | NO: 7) | NO: 5) | NO: 7) | NO: 5) |
| (SEQ ID NO: 1) | | | | | | |
| SBS#74964 | RSADLSE | RSAHLSR | RSADLSE | RSAHLSR | RSADLSE | RSAHLSR |
| tagGGGCCGGGGCCGG | (SEQ ID | (SEQ ID | (SEQ ID | (SEQ ID | (SEQ ID | (SEQ ID |
| GGCCGgggcgtg | NO: 8) | NO: 5) | NO: 8) | NO: 5) | NO: 8) | NO: 5) |
| (SEQ ID NO: 1) | | | | | | |
| SBS#74969 | RSDHLSE | DRSHLAR | RSDHLSE | DRSHLAR | RSDHLSE | DRSHLAR |
| taggGGCCGGGGCCGG | (SEQ ID | (SEQ ID | (SEQ ID | (SEQ ID | (SEQ ID | (SEQ ID |
| GGCCGGggcgtg | NO: 9) | NO: 10) | NO: 9) | NO: 10) | NO: 9) | NO: 10) |
| (SEQ ID NO: 1) | | | | | | |
| SBS#74971 | RSDHLSQ | DNSHRTR | RSDHLSQ | DNSHRTR | RSDHLSQ | DNSHRTR |
| taggGGCCGGGGCCGG | (SEQ ID | (SEQ ID | (SEQ ID | (SEQ ID | (SEQ ID | (SEQ ID |
| GGCCGGggcgtg | NO: 11) | NO: 12) | NO: 11) | NO: 12) | NO: 11) | NO: 12) |
| (SEQ ID NO: 1) | | | | | | |
| SBS#74973 | RNGHLLD | DRSHLAR | RNGHLLD | DRSHLAR | RNGHLLD | DRSHLAR |
| taggGGCCGGGGCCGG | (SEQ ID | (SEQ ID | (SEQ ID | (SEQ ID | (SEQ ID | (SEQ ID |
| GGCCGGggcgtg | NO: 13) | NO: 10) | NO: 13) | NO: 10) | NO: 13) | NO: 10) |
| (SEQ ID NO: 1) | | | | | | |
| SBS#74978 | RNGHLLD | DNSHRTR | RNGHLLD | DNSHRTR | RNGHLLD | DNSHRTR |
| taggGGCCGGGGCCGG | (SEQ ID | (SEQ ID | (SEQ ID | (SEQ ID | (SEQ ID | (SEQ ID |
| GGCCGGggcgtg | NO: 13) | NO: 12) | NO: 13) | NO: 12) | NO: 13) | NO: 12) |
| (SEQ ID NO: 1) | | | | | | |
| SBS#74979 | RSAHLSE | DNSHRTR | RSAHLSE | DNSHRTR | RSAHLSE | DNSHRTR |
| taggGGCCGGGGCCGG | (SEQ ID | (SEQ ID | (SEQ ID | (SEQ ID | (SEQ ID | (SEQ ID |
| GGCCGGggcgtg | NO: 14) | NO: 12) | NO: 14) | NO: 12) | NO: 14) | NO: 12) |
| (SEQ ID NO: 1) | | | | | | |
| SBS#74983 | RSAHLSR | DRSDLSR | RSAHLSR | DRSDLSR | RSAHLSR | DRSDLSR |
| tagggGCCGGGGCCGG | (SEQ ID | (SEQ ID | (SEQ ID | (SEQ ID | (SEQ ID | (SEQ ID |
| GGCCGGGgcgtg | NO: 5) | NO: 3) | NO: 5) | NO: 3) | NO: 5) | NO: 3) |
| (SEQ ID NO: 1) | | | | | | |
| SBS#74984 | RSDHLSR | DWTTRRR | RSDHLSR | DWTTRRR | RSDHLSR | DWTTRRR |
| tagggGCCGGGGCCGG | (SEQ ID | (SEQ ID | (SEQ ID | (SEQ ID | (SEQ ID | (SEQ ID |
| GGCCGGGgcgtg | NO: 15) | NO: 16) | NO: 15) | NO: 16) | NO: 15) | NO: 16) |
| (SEQ ID NO: 1) | | | | | | |
| SBS#74986 | RSAHLSR | HRKSLSR | RSAHLSR | HRKSLSR | RSAHLSR | HRKSLSR |
| tagggGCCGGGGCCGG | (SEQ ID | (SEQ ID | (SEQ ID | (SEQ ID | (SEQ ID | (SEQ ID |
| GGCCGGGgcgtg | NO: 5) | NO: 17) | NO: 5) | NO: 17) | NO: 5) | NO: 17) |
| (SEQ ID NO: 1) | | | | | | |
| SBS#74987 | RSAHLSR | DSSDRKK | RSAHLSR | DSSDRKK | RSAHLSR | DSSDRKK |
| tagggGCCGGGGCCGG | (SEQ ID | (SEQ ID | (SEQ ID | (SEQ ID | (SEQ ID | (SEQ ID |
| GGCCGGGgcgtg | NO: 5) | NO: 18) | NO: 5) | NO: 18) | NO: 5) | NO: 18) |
| (SEQ ID NO: 1) | | | | | | |
| SBS#74988 | RSAHLSR | DSSTRRR | RSAHLSR | DSSTRRR | RSAHLSR | DSSTRRR |
| tagggGCCGGGGCCGG | (SEQ ID | (SEQ ID | (SEQ ID | (SEQ ID | (SEQ ID | (SEQ ID |
| GGCCGGGgcgtg | NO: 5) | NO: 19) | NO: 5) | NO: 19) | NO: 5) | NO: 19) |
| (SEQ ID NO: 1) | | | | | | |
| SBS#74997 | RSAHLSR | RSDDRKT | RSAHLSR | RSDDRKT | RSAHLSR | RSDDRKT |
| taggggCCGGGGCCGG | (SEQ ID | (SEQ ID | (SEQ ID | (SEQ ID | (SEQ ID | (SEQ ID |
| GGCCGGGGcgtg | NO: 5) | NO: 20) | NO: 5) | NO: 20) | NO: 5) | NO: 20) |
| (SEQ ID NO: 1) | | | | | | |
| SBS#74998 | RSAHLSR | RSADRKT | RSAHLSR | RSADRKT | RSAHLSR | RSADRKT |
| taggggCCGGGGCCGG | (SEQ ID | (SEQ ID | (SEQ ID | (SEQ ID | (SEQ ID | (SEQ ID |
| GGCCGGGGcgtg | NO: 5) | NO: 21) | NO: 5) | NO: 21) | NO: 5) | NO: 21) |
| (SEQ ID NO: 1) | | | | | | |
| SBS#75001 | RSAHLSR | RNADRIT | RSAHLSR | RNADRIT | RSAHLSR | RNADRIT |
| taggggCCGGGGCCGG | (SEQ ID | (SEQ ID | (SEQ ID | (SEQ ID | (SEQ ID | (SEQ ID |
| GGCCGGGGcgtg | NO: 5) | NO: 22) | NO: 5) | NO: 22) | NO: 5) | NO: 22) |
| (SEQ ID NO: 1) | | | | | | |
| SBS#75003 | RSAHLSR | RRATLLD | RSAHLSR | RRATLLD | RSAHLSR | RRATLLD |
| taggggCCGGGGCCGG | (SEQ ID | (SEQ ID | (SEQ ID | (SEQ ID | (SEQ ID | (SEQ ID |
| GGCCGGGGcgtg | NO: 5) | NO: 23) | NO: 5) | NO: 23) | NO: 5) | NO: 23) |
| (SEQ ID NO: 1) | | | | | | |
| SBS#75023 | RSDTLSV | DTSTRTK | RSDTLSV | DTSTRTK | RSDTLSV | DTSTRTK |
| cacGCCCCGGCCCCGG | (SEQ ID | (SEQ ID | (SEQ ID | (SEQ ID | (SEQ ID | (SEQ ID |
| CCCCGgccccta | NO: 24) | NO: 25) | NO: 24) | NO: 25) | NO: 24) | NO: 25) |
| (SEQ ID NO: 2) | | | | | | |
| SBS#75027 | RNADRIT | HRKSLSR | RNADRIT | HRKSLSR | RNADRIT | RNADRIT |
| cacGCCCCGGCCCCGG | (SEQ ID | (SEQ ID | (SEQ ID | (SEQ ID | (SEQ ID | (SEQ ID |
| CCCCGgccccta | NO: 22) | NO: 17) | NO: 22) | NO: 17) | NO: 22) | NO: 22) |
| (SEQ ID NO: 2) | | | | | | |
| SBS#75031 | RSADRKT | HRKSLSR | RSADRKT | HRKSLSR | RSADRKT | HRKSLSR |
| cacGCCCCGGCCCCGG | (SEQ ID | (SEQ ID | (SEQ ID | (SEQ ID | (SEQ ID | (SEQ ID |
| CCCCGgccccta | NO: 21) | NO: 17) | NO: 21) | NO: 17) | NO: 21) | NO: 17) |
| (SEQ ID NO: 2) | | | | | | |
| SBS#75032 | RSATLSE | HRKSLSR | RSATLSE | HRKSLSR | RSATLSE | HRKSLSR |
| cacGCCCCGGCCCCGG | (SEQ ID | (SEQ ID | (SEQ ID | (SEQ ID | (SEQ ID | (SEQ ID |
| CCCCGgccccta | NO: 26) | NO: 17) | NO: 26) | NO: 17) | NO: 26) | NO: 17) |
| (SEQ ID NO: 2) | | | | | | |
| SBS#75055 | RSADRKT | DSSTRRR | RSADRKT | DSSTRRR | RSADRKT | DSSTRRR |
| cacGCCCCGGCCCCGG | (SEQ ID | (SEQ ID | (SEQ ID | (SEQ ID | (SEQ ID | (SEQ ID |
| CCCCGgccccta | NO: 21) | NO: 19) | NO: 21) | NO: 19) | NO: 21) | NO: 19) |
| (SEQ ID NO: 2) | | | | | | |
| SBS#75078 | RSADLSE | HHRSLHR | RSADLSE | HHRSLHR | RSADLSE | HHRSLHR |
| cacGCCCCGGCCCCGG | (SEQ ID | (SEQ ID | (SEQ ID | (SEQ ID | (SEQ ID | (SEQ ID |
| CCCCGgccccta | NO: 8) | NO: 27) | NO: 8) | NO: 27) | NO: 8) | NO: 27) |
| (SEQ ID NO: 2) | | | | | | |
| SBS#75090 | RSDHLSE | TSSDRTK | RSDHLSE | TSSDRTK | RSDHLSE | TSSDRTK |
| cacgCCCCGGCCCCGG | (SEQ ID | (SEQ ID | (SEQ ID | (SEQ ID | (SEQ ID | (SEQ ID |
| CCCCGGccccta | NO: 9) | NO: 28) | NO: 9) | NO: 28) | NO: 9) | NO: 28) |
| (SEQ ID NO: 2) | | | | | | |
| SBS#75105 | DRSHLTR | DSSTRKT | DRSHLTR | DSSTRKT | DRSHLTR | DSSTRKT |
| cacgcCCCGGCCCCG | (SEQ ID | (SEQ ID | (SEQ ID | (SEQ ID | (SEQ ID | (SEQ ID |
| GCCCCGGCcccta | NO: 29) | NO: 30) | NO: 29) | NO: 30) | NO: 29) | NO: 30) |
| (SEQ ID NO: 2) | | | | | | |
| SBS#75109 | DKRDLAR | RSADRKT | DKRDLAR | RSADRKT | DKRDLAR | RSADRKT |
| cacgccCCGGCCCCGG | (SEQ ID | (SEQ ID | (SEQ ID | (SEQ ID | (SEQ ID | (SEQ ID |
| CCCCGGCCccta | NO: 31) | NO: 21) | NO: 31) | NO: 21) | NO: 31) | NO: 21) |
| (SEQ ID NO: 2) | | | | | | |
| SBS#75114 | ERGTLAR | RSADRKT | ERGTLAR | RSADRKT | ERGTLAR | RSADRKT |
| cacgccCCGGCCCCGG | (SEQ ID | (SEQ ID | (SEQ ID | (SEQ ID | (SEQ ID | (SEQ ID |
| CCCCGGCCccta | NO: 7) | NO: 21) | NO: 7) | NO: 21) | NO: 7) | NO: 21) |
| (SEQ ID NO: 2) | | | | | | |
| SBS#75115 | ERRDLRR | RSADRKT | ERRDLRR | RSADRKT | ERRDLRR | RSADRKT |
| cacgccCCGGCCCCGG | (SEQ ID | (SEQ ID | (SEQ ID | (SEQ ID | (SEQ ID | (SEQ ID |
| CCCCGGCCccta | NO: 32) | NO: 21) | NO: 32) | NO: 21) | NO: 32) | NO: 21) |
| (SEQ ID NO: 2) | | | | | | |

All repressing transcription factors (TFs) are operably linked to a repression domain (e.g., KRAB) to form TFs that repress DUX4, C9orf72 or Ube3a-ATS. The TFs are transfected into mouse Neuro2a cells. After 24 hours, total RNA is extracted and the expression of DUX4, C9orf72 or Ube3a-ATS and two reference genes (ATP5b, RPL38) is monitored using real-time RT-qPCR.

The TFs are found to be effective in repressing DUX4, C9orf72 or Ube3a-ATS expression with a diversity of dose-response and target gene repression activity. In particular, C9orf72 ZFP-TF repressors (comprising the ZFPs of Table 1) and a transcriptional repression domain (KRAB) were introduced into C9021 cells obtained from ALS institute at Columbia University. The line contains 5 G4C2 repeat (SEQ ID NO: 35) on its normal allele and more than 145 repeats on its expanded allele. The wildtype cell line was NDS00035 obtained from NINDS and it contained two G4C2 (SEQ ID NO: 36) repeats on each allele. mRNA transfection was performed using 96-well Shuttle Nucleofector system from Lonza. 1, 3, 10, 30, 100, and 300 ng of ZFP mRNA per 40,000 cells were transfected using Amaxa P2 Primary Cells Nucleofector kit using CA-137 program. After overnight incubation, a Cells-to-Ct kit (Thermo Fisher Scientific) was used to generate cDNA from transfected cells followed by gene expression analysis using qRT-PCR.

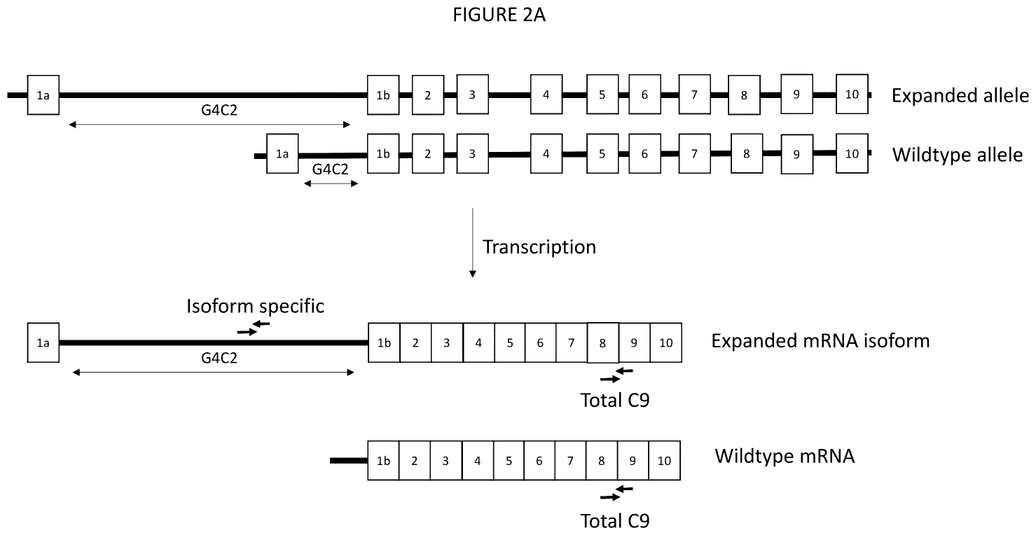

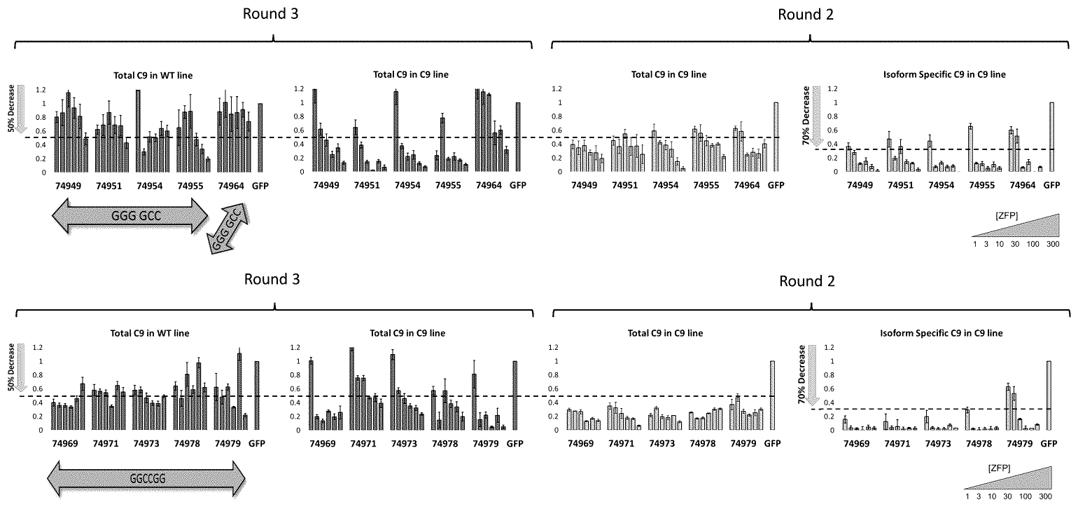

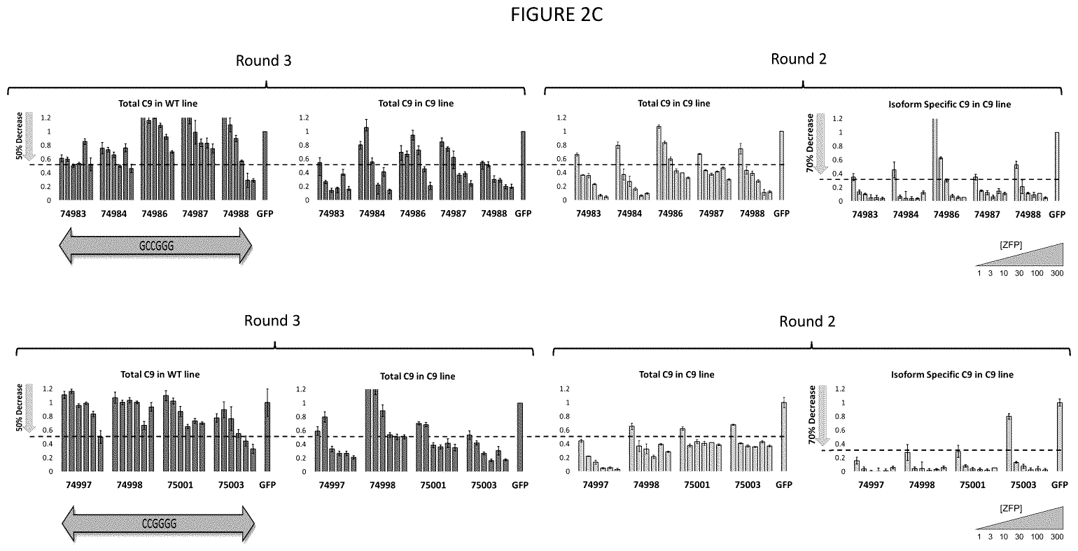

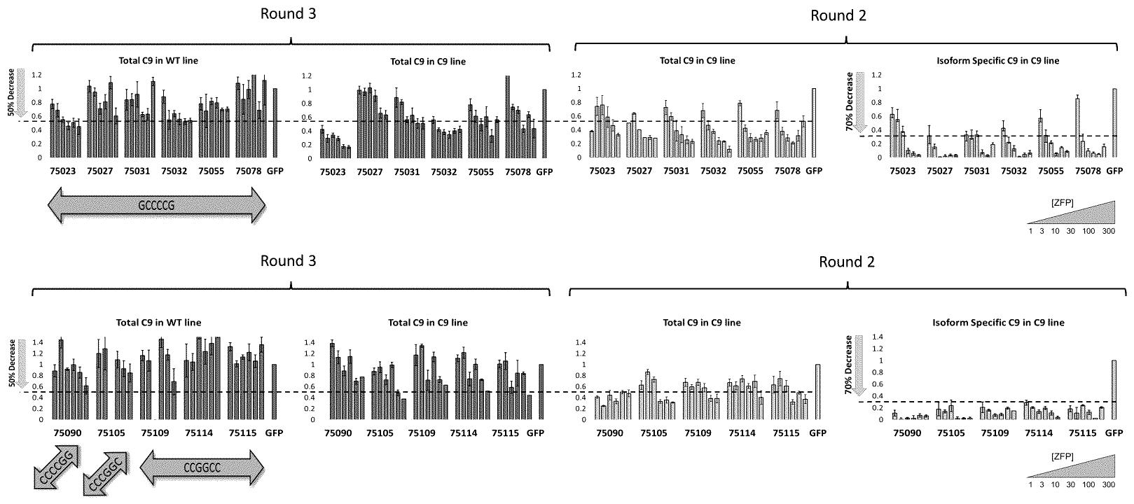

Exemplary results are shown in FIG. 2, where repression of wild-type and mutant alleles was observed. In addition to investigating total C9orf72 repression, an “isoform specific” RT-PCR assay was used which detected a longer mRNA message (comprising intron 1A) versus a wildtype (shorter) mRNA message. The “isoform specific assay” detects the repression of the longer mRNA species (see FIG. 2A). The longer mRNA isoform is produced predominantly by the expanded (diseased) allele, although it is also produced by a wildtype allele to a much lesser extent. The assay uses two primer/probe sets, wherein the first set is used in the isoform specific assay, and targets the intronic region la which is present in the diseased or expanded isoform (see FIG. 2A). By using this assay in C9 lines, we showed that ZFPs, such as 75114 and 75115, represses the diseased isoform by more than 70% (FIG. 2B through 2D). Thus, reduction of expression of the longer mRNA isoform is an indication of repression of mRNA expression from the expanded (diseased) allele.

In order to evaluate repression of the wildtype isoform, a primer/probe set denoted as ‘Total C9’ (FIG. 2A) was used which detects mRNAs encoding exonic regions 8 and 9. These regions were present in both the disease and wildtype isoforms, thus the repression of C9orf72 expression observed in the C9 lines in the Total C9 assay (FIG. 2B through 2D) represents repression of expression of both the disease and wildtype isoforms in response to ZFP treatment. Thus, total C9orf72 mRNA levels in wildtype lines, comprising predominantly the wildtype isoform, was analyzed where retention of more than 50% of the wildtype isoform was observed in response to ZFP-TF treatment.

Similarly, all activating TFs are operably linked to an activation domain (e.g., HSV VP16) to form TFs that activate paternal UBE34, SMCHD1, SMN1 or SMN2. The ZFP TFs are transfected into mouse Neuro2a or fibroblast cells. After 24 hours, total RNA is extracted and the expression of UBE34, SMCHD1, SMN1 or SMN2 and two reference genes is monitored using real-time RT-qPCR.

The TFs are found to be effective in repressing UBE34, SMCHD1, SMN1 or SMN2 expression with a diversity of dose-response and target gene repression activity.

### Example 2: Specificity of C9orf72 Repression

The global specificity of the ZFP-TFs shown in Table 1 was evaluated by microarray analysis in C9021 cells. In brief, 100 ng of ZFP-TF encoding mRNA was transfected into 150,000 C9021 cells in biological quadruplicate. After 24 hours, total RNA was extracted and processed via the manufacturer's protocol (Affymetrix Genechip MTA1.0). Robust Multi-array Average (RMA) was used to normalize raw signals from each probe set. Analysis was performed using Transcriptome Analysis Console 3.0 (Affymetrix) with the “Gene Level Differential Expression Analysis” option. ZFP-transfected samples were compared to samples that had been treated with an irrelevant ZFP-TF (that does not bind to C9orf72 target site). Change calls are reported for transcripts (probe sets) with a >2 fold difference in mean signal relative to control, and a P-value <0.05 (one-way ANOVA analysis, unpaired T-test for each probeset).

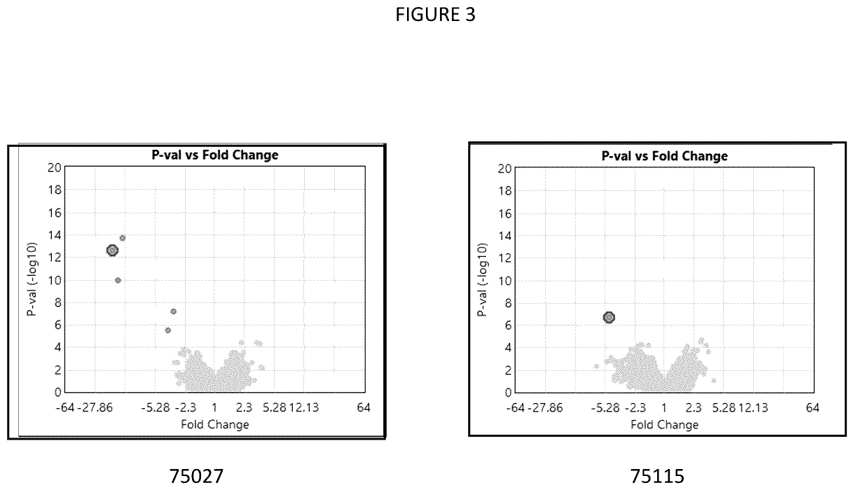

As shown in FIG. 3, SBS #75027 repressed 4 genes in addition to C9orf72 (shown circled) while SBS #75115 repressed only C9orf72. These results demonstrate that the ZFP-TFs are highly specific for C9orf72.

### Example 3: Gene Modulation in Mouse Neurons

All repressors targeted to mouse DUX4, C9orf72 or Ube3a-ATS are cloned into rAAV2/9 vectors using a CMV promoter to drive expression. Virus is produced in HEK293T cells, purified using a CsCl density-gradient, and titered by real time qPCR according to methods known in the art. The purified virus is used to infect cultured primary mouse cortical neurons at 3E5, 1E5, 3E4, and 1E4 VG/cell. After 7 days, total RNA is extracted and the expression of DUX4, C9orf72 or Ube3a-ATS and two reference genes (ATP5b, EIF4a2) was monitored using real-time RT-qPCR.

All TF-encoding AAV vectors are found to effectively repress mouse their targets over a broad range of infected doses, with some ZFPs reducing the target by greater than 95% at multiple doses. In contrast, no gene repression is observed for a rAAV2/9 CMV-GFP virus tested at equivalent doses, or mock-treated neurons.

Thus, genetic modulators (e.g., repressors or activators) as described herein, are functional repressors or activators when formulated as plasmids, in mRNA form, in Ad vectors and/or in AAV vectors.

### Example 4: In Vivo Gene Repression Driven by AAV-Delivered TFs

TFs are delivered to the mouse hippocampus to evaluate repression of DUX4, C9orf72 or Ube3a-ATS in vivo. In brief, a total dose of 8E9 VGs of rAAV2/9-CMV-ZFP-TF per hemisphere is administered by stereotactic injection via dual, bilateral 2 μL injections. The animals are sacrificed five weeks post-injection and each hemisphere is sectioned into three pieces for analysis. DUX4, C9orf72 or Ube3a-ATS and ZFP-TF expression is analyzed by real time RT-qPCR and normalized to the geometric mean of three housekeeping genes (ATP5b, EIF4a2 and GAPDH).

The data show that, relative to the PBS treated cohort, the TFs are able to repress their targets efficiently.

In addition, the genetic modulators are cloned into an AAV vector (AAV2/9, or variants thereof) for example with the SYN1 promoter or CMV promoter, essentially as described in U.S. Publication No. 20180153921. AAV vectors used included: a vector with a SYN1 promoter driving expression of repressors comprising one or more ZFP-TFs comprising the ZFPs of Table 1. Two or more ZFP-TFs are linked by suitable IRES or 2A peptide sequences (e.g., T2A or P2A) and administered to human and non-human primate subjects with or without ALS or FTD at dosages of 1E10 to 1E13 (e.g., 6E11) vg/hemisphere (to each hemisphere), preferably to the hippocampus. Some subjects receive one or more additional dosages at any time.

The results show that genetic repressors as described herein delivered by AAV to the brain lead to reduction in expression of the target gene (e.g., C9orf72) and to amelioration of symptoms of ALS or FTD subjects.

All patents, patent applications and publications mentioned herein are hereby incorporated by reference for all purposes in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A genetic modulator of a C9orf72 gene, the modulator comprising
a DNA-binding domain that binds to a target site of at least 12 nucleotides in the C9orf72 gene; and
a transcriptional regulatory domain or nuclease domain.

2. The genetic modulator of claim 1, wherein the DNA-binding domain comprises
a zinc finger protein (ZFP), a TAL-effector domain protein (TALE) or single guide RNA.

3. The genetic modulator of claim 1, wherein the transcriptional regulatory domain comprises a repression domain or activation domain.

4. A polynucleotide encoding the genetic modulator according to claim 1.

5. A gene delivery vehicle comprising the polynucleotide according to claim 4.

6. The gene delivery vehicle of claim 5, wherein the gene delivery vehicle comprises an AAV vector.

7. A pharmaceutical composition comprising one or more polynucleotides according to claim 4.

8. The pharmaceutical composition of claim 7, wherein the genetic modulator comprises a nuclease domain and the genetic modulator cleaves the C9orf72 gene.

9. The pharmaceutical composition of claim 8, further comprising a donor molecule that is integrated into the cleaved C9orf72 gene.

10. An isolated cell comprising one or more genetic modulators according to claim 1.

11. An isolated cell comprising one or more polynucleotides according to claim 4.

12. A method of modulating C9orf72 gene expression in a cell, the method comprising administering one or more genetic modulators according to claim 1 to a cell.

13. The method of claim 12, wherein C9orf72 gene expression is repressed.

14. The method of claim 13, wherein both sense and antisense C9orf72 gene expression is repressed.

15. The method of claim 14, wherein the administration is intracerebroventricular, intrathecal, intracranial, retro-orbital (RO), intravenous, intranasal or intracisternal.

16. A method of treating and/or preventing Amyotrophic Lateral Sclerosis (ALS) or Frontotemporal dementia (FTD) in a subject, the method comprising repressing C9orf72 expression according to the method of claim 15.

17. A kit comprising one or more of gene delivery vehicles according to claim 6 and/or instructions for use.
